SEQUENCE LISTING (34 sequences)

SEQ ID NO: 1 - protein, 19 aa, Artificial Sequence
  synthetic peptide based on the silaffin repetitive unit
SSKKSGSYSGSKGSKRRIL

SEQ ID NO: 2 - protein, 5 aa, Artificial Sequence
  truncated silaffin peptide I
KRRIL

SEQ ID NO: 3 - protein, 5 aa, Artificial Sequence
  truncated silaffin peptide generic sequence
  Xaa (4)..(4): peptide bond or Ile moiety
RRIXL

SEQ ID NO: 4 - protein, 4 aa, Artificial Sequence
  truncated silaffin peptide II
RRIL

SEQ ID NO: 5 - protein, 5 aa, Artificial Sequence
  truncated silaffin peptide III
RRIIL

SEQ ID NO: 6 - protein, 9 aa, Artificial Sequence
  truncated silaffin peptide dimer I
RRILKRRIL

SEQ ID NO: 7 - protein, 9 aa, Artificial Sequence
  truncated silaffin peptide dimer II
  Xaa (5)..(5): Lys conjugated to the preceding Leu via its epsilon amino group
RRILXRRIL

SEQ ID NO: 8 - protein, 10 aa, Artificial Sequence
  truncated silaffin peptide dimer III
  Xaa (6)..(6): Lys conjugated to the preceding Leu via its epsilon amino group
RRIILXRRIL

SEQ ID NO: 9 - protein, 10 aa, Artificial Sequence
  truncated silaffin peptide dimer IV
RRILKRRIIL

SEQ ID NO: 10 - protein, 10 aa, Artificial Sequence
  truncated silaffin peptide dimer V
RRIILKRRIL

SEQ ID NO: 11 - protein, 11 aa, Artificial Sequence
  truncated silaffin peptide dimer VI
RRIILKRRIIL

SEQ ID NO: 12 - protein, 10 aa, Artificial Sequence
  truncated silaffin peptide dimer VII
  Xaa (5)..(5): Lys conjugated to the preceding Leu via its epsilon amino group
RRILXRRIIL

SEQ ID NO: 13 - protein, 11 aa, Artificial Sequence
  truncated silaffin peptide dimer VIII
  Xaa (6)..(6): Lys conjugated to the preceding Leu via its epsilon amino group
RRIILXRRIIL

SEQ ID NO: 14 - protein, 4 aa, Artificial Sequence
  alternative structure for rod formation
IIIK

SEQ ID NO: 15 - protein, 11 aa, Artificial Sequence
  divalent conjugate generic structure
  Xaa (4)..(4): peptide bond or Ile
  Xaa (6)..(6): any amino acid
  Xaa (10)..(10): peptide bond or Ile
RRIXLXRRIXL

SEQ ID NO: 16 - protein, 9 aa, Artificial Sequence
  truncated silaffin peptide dimer IX
LIRRKRRIL

SEQ ID NO: 17 - protein, 9 aa, Artificial Sequence
  truncated silaffin dimer X
  Xaa (5)..(5): Lys conjugated to the preceding Leu via its epsilon amino group
LIRRXRRIL

SEQ ID NO: 18 - protein, 10 aa, Artificial Sequence
  truncated silaffin peptide XIV
RRILKLIIRR

SEQ ID NO: 19 - protein, 4 aa, Artificial Sequence
  reverse motif
LIRR

SEQ ID NO: 20 - protein, 11 aa, Artificial Sequence
  divalent generic structure II
  Xaa (2)..(2): peptide bond or Ile
  Xaa (6)..(6): any amino acid residue
  Xaa (10)..(10): peptide bond or Ile
LXIRRXRRIXL

SEQ ID NO: 21 - protein, 5 aa, Artificial Sequence
  alternative reverse motif
LIIRR

SEQ ID NO: 22 - protein, 265 aa, Cylindrotheca fusiformis
MKLTAIFPLLFTAVGYCAAQSIADLAAANLSTEDSKSAQLISADSSDDASDSSVESVDAASSDVSGSSVESVDVSGSSLESVDVSGSSLESVDDSSEDSEEEELRILSSKKSGSYYSYGTKKSGSYSGYSTKKSASRRILSSKKSGSYSGYSTKKSGSRRILSSKKSGSYSGSKGSKRRILSSKKSGSYSGSKGSKRRNLSSKKSGSYSGSKGSKRRILSSKKSGSYSGSKGSKRRNLSSKKSGSYSGSKGSKRRILSGGLRGSM

SEQ ID NO: 23 - protein, 5 aa, Artificial Sequence
  general forward sequence motif for silica precipitation
  Xaa (3)..(3): any hydrophobic amino acid moiety
  Xaa (4)..(4): peptide bond or any hydrophobic amino acid moiety
RRXXL

SEQ ID NO: 24 - protein, 5 aa, Artificial Sequence
  general reverse sequence motif for silica precipitation
  Xaa (2)..(2): any hydrophobic amino acid moiety
  Xaa (3)..(3): peptide bond or any hydrophobic amino acid moiety
LXXRR

SEQ ID NO: 25 - protein, 11 aa, Artificial Sequence
  general divalent peptide I
  Xaa (3)..(3): any hydrophobic amino acid moiety
  Xaa (4)..(4): peptide bond or any hydrophobic amino acid moiety
  Xaa (6)..(6): any peptide moiety of not more than five consecutive amino acid moieties
  Xaa (9)..(9): any hydrophobic amino acid moiety
  Xaa (10)..(10): ppetide bond or any hydrophobic amino acid moiety
RRXXLXRRXXL

SEQ ID NO: 26 - protein, 11 aa, Artificial Sequence
  general divalent peptide II
  Xaa (2)..(2): any hydrophobic amino acid moiety
  Xaa (3)..(3): peptide bond or any hydrophobic amino acid moiety
  Xaa (6)..(6): any peptide moiety of not more than five consecutive amino acid moieties
  Xaa (9)..(9): any hydrophobic amino acid moiety
  Xaa (10)..(10): ppetide bond or any hydrophobic amino acid moiety
LXXRRXRRXXL

SEQ ID NO: 27 - protein, 9 aa, Artificial Sequence
  branched sequence
  Xaa (5)..(5): Lys bearing a sequence of four or five consecutive amino acid residues conjugated to the epsilon amino group of the Lys
LIRRXRRIL

SEQ ID NO: 28 - protein, 11 aa, Artificial Sequence
  general divalent peptide III
  Xaa (3)..(3): any hydrophobic amino acid moiety
  Xaa (4)..(4): peptide bond or any hydrophobic amino acid moiety
  Xaa (6)..(6): any peptide moiety of not more than five consecutive amino acid moieties
  Xaa (8)..(8): any hydrophobic amino acid moiety
  Xaa (9)..(9): peptide bond or any hydrophobic amino acid moiety
RRXXLXLXXRR

SEQ ID NO: 29 - protein, 10 aa, Artificial Sequence
  truncated silaffin peptide XV
  Xaa (6)..(6): Lys conjugated to the preceding Leu via its epsilon amino group
RRILLXRRIL

SEQ ID NO: 30 - protein, 10 aa, Artificial Sequence
  truncated silaffin peptide XVI
  Xaa (6)..(6): Lys conjugated to the preceding Leu via its epsilon amino group
RRIILXRRLL

SEQ ID NO: 31 - protein, 9 aa, Artificial Sequence
  truncated silaffin PEG conjugate
  Xaa (5)..(5): polyethylene glycol  (PEG)3-conjugated to the preceding Leu and tehsubsequent Arg
RRILXRRIL

SEQ ID NO: 32 - protein, 28 aa, Artificial Sequence
  silaffin peptide
SIINFEKLCSSKKSGSYSGSKGSKRRIL

SEQ ID NO: 33 - protein, 5 aa, Artificial Sequence
  truncated silaffin peptide XVII
RRILL

SEQ ID NO: 34 - protein, 4 aa, Artificial Sequence
  truncated silaffin peptide XVIII
RRLL

(19) United States
(12) Patent Application Publication
(10) Pub. No.: US 2021/0106679 A1
(43) Pub. Date: Apr. 15, 2021

(54) Silaffin Silica Particle Adjuvant

(71) Applicant: UNIVERSITÄT WIEN, Wien (AT)

(72) Inventors: Christian Friedrich Wilhelm Becker, Klosterneuburg (AT); Meder Kamalov, Wien (AT)

(73) Assignee: UNIVERSITÄT WIEN, Wien (AT)

(21) Appl. No.: 16/606,576

(22) PCT Filed: Apr. 19, 2018
(86) PCT No.: PCT/EP2018/060052

§ 371 Date: Oct. 18, 2019

(30) Foreign Application Priority Data
Apr. 20, 2017 (EP) 17167306.4

(51) Int. Cl. A61K 39/39; C07K 17/14

(52) U.S. Cl. CPC A61K 39/39; A61K 2039/55516; C07K 17/14

(57) ABSTRACT

The present invention relates to a vaccine composition V comprising a peptidic entity A comprising one or more sequence motifs A1 of the sequence RR-X^{1a}-X^{1b}-L (SEQ ID NO: 23) wherein X^{1a} is a hydrophobic amino acid moiety and X^{1b} is a peptide bond or a hydrophobic amino acid moiety, or an analogue thereof, an antigen of interest B and silica particles C embracing A and B. Further, the present invention relates to the peptidic entity A usable for such purpose and pharmaceutical and non-pharmaceutical uses of the vaccine composition V.

The present invention relates to a vaccine composition V comprising a peptidic entity A comprising one or more sequence motifs A1 of the sequence RR-X^{1a}-X^{1b}-L (SEQ ID NO: 23) wherein X^{1a} is a hydrophobic amino acid moiety and X^{1b} is a peptide bond or a hydrophobic amino acid moiety, or an analogue thereof, an antigen of interest B and silica particles C embracing A and B. Further, the present invention relates to the peptidic entity A usable for such purpose and pharmaceutical and non-pharmaceutical uses of the vaccine composition V.

Vaccines provide longterm resistance against diseases by generating a strong immune response to disease antigens. Unfortunately, many vaccines have rather poor activity when administered as plain antigen. Therefore, adjuvants are added to enhance the immune response. In other words, adjuvants are often added to the antigens in order to boost their effectiveness by heightening the resultant immune response or vaccine immunogenicity (S. G. Reed, M. T. Orr and C. B. Fox, Wat. Med., 2013, 19, 1597-1608).

Traditional vaccines, made of attenuated pathogens, have had an immense impact on public health by eradicating or nearly eliminating a number of deadly diseases such as small pox and polio. Traditional vaccines are often sufficiently immunogenic on their own, although adjuvants can be added to some in order to further enhance the effectiveness. The limited specificity of the immune response against traditional vaccines makes them unsuitable for a number of challenging disease targets such as malaria, metastatic cancer and Human Immunodeficiency Virus (HIV) 0.7-9 (S. G. Reed, M. T. Orr and C. B. Fox, Wat. Med., 2013, 19, 1597-1608)

Subunit vaccines, unlike traditional vaccines, consist of recombinant or synthetic antigens (i.e., antigen in its poorer form), such as peptides (as peptidic antigens), which can be carefully chosen from the mechanistic analysis of the disease progression. Subunit vaccines offer considerable advantages over the traditional vaccines in terms of specificity, safety and cost of production (S. G. Reed, M. T. Orr and C. B. Fox, Wat. Med., 2013, 19, 1597-1608). However, subunit vaccines often show limited immunogenicity and therefore low effectiveness rates, which are predominantly due to rapid degradation of the vaccines by the host organism. Addition of adjuvants is therefore essential in order to maximize the long-lasting immune responses to subunit vaccines (S. G. Reed, M. T. Orr and C. B. Fox, Wat. Med., 2013, 19, 1597-1608). Some subunit vaccines, e.g. human papilloma virus, can successfully elicit protective antibody responses using only alum as an adjuvant, which is known to increase the antibody response (S. G. Reed, S. Bertholet, R. N. Coler and M. Friede, Trends Immunol., 2009, 30, 23-32).

In particular vaccines based on short peptide sequences are of interest as these, in principle, enable treatment of challenging disease targets such as malaria, metastatic cancer and HIV (F. Zavala, J. Exp. Med., 1987, 166, 1591-1596; D. J. Schwartzentruber, D. H. Lawson, J. M. Richards, R. M. Conry, D. M. Miller, J. Treisman, F. Gailani, L. Riley, K. Conlon, B. Pockaj, K. L., Kendra, R. L. White, R. Gonzalez, T. M. Kuzel, B. Curti, P. D. Leming, E. D. Whitman, J. Balkissoon, D. S. Reintgen, H. Kaufman, F. M. Marincola, M. J. Merino, S. A. Rosenberg, P. Choyke, D. Vena and P. Hwu, N. Engl. J. Med., 2011, 364, 2119-2127; I. M. Belyakov, M. A. Derby, J. D. Ahlers, B. L. Kelsall, P. Earl, B. Moss, W. Strober and J. A. Berzofsky, Proc. Nat. Acad. Sci., 1998, 95, 1709-1714). Effectiveness is however limited. Thus, such vaccines based on short peptide sequences are often weakly immunogenic and require adjuvants for boosted effectiveness (Y. Wen and J. H. Collier, Curr. Opin. Immunol., 2015, 35, 73-79).

Summarized, the next generation of subunit vaccines (i.e., typically bearing a molecular weight of not more than 5000 Da, and/or, in case of a peptide vaccine being a short peptidic entity of (approximately) 5 to 20 amino acid moieties), aimed at diseases such as HIV, tuberculosis and cancer, might require not only very strong and long-lasting antibody responses such as seen for alum but also a potent adaptive immunity based on other pathways such as helper (CD4+) and cytotoxic (CD8+) T-cell responses (S. G. Reed, S. Bertholet, R. N. Coler and M. Friede, Trends Immunol., 2009, 30, 23-32). Given that alum induces a Th-2 based immune response but no cytotoxic responses, it is unlikely to result in the desired immunity and therefore novel adjuvants and formulations are needed (S. G. Reed, S. Bertholet, R. N. Coler and M. Friede, Trends Immunol., 2009, 30, 23-32). Also a number of other substances, such as saponins and different forms of Freunds adjuvant, have been evaluated as potential adjuvants. However, all of these bear significant drawbacks.

Peptide assemblies, such as nanofibers and nanoparticles, can generally be used as adjuvants for vaccines based on short peptides. Multi-valencies and the morphological properties of these assemblies have been employed in vaccine synthesis by attaching different peptide antigens to singular self-assembling domains. The self-assembly of the resulting fusion peptides has been able to elicit strong antibody responses without additional adjuvants (F. Zavala, J. Exp. Med., 1987, 166, 1591-1596). However, the heterogeneous nature of peptide assemblies renders their properties difficult to control, while their amyloid-like nature requires careful analysis of their inherent pathogenicity and undesired toxicity (F. Zavala, J. Exp. Med., 1987, 166, 1591-1596). Accordingly, peptide-based adjuvants also have significant drawbacks.

Accordingly, further alternative adjuvants have been considered. For example, it has been found that silica particles, in particular mesoporous silica particles (MSPs), may serve as usable adjuvants. Such MSPs provide great stability for and controlled release of the associated antigens. (K. T. Mody, A. Popat, D. Mahony, A. S. Cavallaro, C. Yu and N. Mitter, Nanoscale, 2013, 5, 5167-5179). Most notably, MSPs have been shown to be potentially effective anti-cancer adjuvants as they can inhibit the in vivo tumour growth when compared with vaccination with alum or without an adjuvant (6X. Wang, X. Li, A. Ito, Y. Watanabe, Y. Sogo, N. M. Tsuji and T. Ohno, Angew. Chem., 2016, 128, 1931-1935). WO 2008/140472 describes alternative autosilification moieties.

Therefore, silica particles, in particular mesoporous silica particles (MSPs), are promising adjuvants.

However, the use of such silica particles is still hampered by the lack of means for efficient loading antigens of interest into the silica particles. Further, suitable silica particles are not easily synthetically accessible. If the silica particles are prepared before loading with the antigen, the loading yield is rather poor. Encapsulation of the antigens within the silica particles typically takes place via absorption into particles by simple stirring in a suspension. Therefore, the efficiency of antigen loading into and onto the silica particles as performed in the art so far is not reliable as it is highly dependent on the biophysical properties of antigens (K. T. Mody, A. Popat, D. Mahony, A. S. Cavallaro, C. Yu and N. Mitter, Nanoscale, 2013, 5, 5167-5179). The preparation of silica particles as such is typically carried out under rather harsh chemical conditions that would harm the integrity of most antigens. So far, silica particles are typically synthesized using organic templates, which subsequently have to be removed under harsh reaction conditions.

Amorphous silica together with the associated organic matter make up the highly elaborate nanopatterns on the surfaces of diatoms (R. E. Hecky, K. Mopper, P. Kilham and E. T. Degens, Mar. Biol., 1973, 19, 323-331). Silica particles can in general also be obtained by precipitation of silica by means of proteins of diatoma. Diatomic biosilica has remarkable mechanical stability and forms under mild aqueous conditions, which is in contrast to common industrial syntheses of silica that require harsh temperature and pH conditions (R. Wetherbee, Science, 2002, 298, 547-547; N. Kröger, R. Deutzmann and M. Sumper, J. Biol. Chem., 2001, 276, 26066-26070; N. Kröger, R. Deutzmann and M. Sumper, Science, 1999, 286, 1129-1132). Different classes of biomolecules have been identified in diatomic biosilica and these include polyamines, polysaccharides and a range proteins, such as frustulins, pleuralins, silacidins, cingulins and silaffins (R. Wetherbee, Science, 2002, 298, 547-547). The latter along with polyamines have been shown to be directly involved in the silica biogenesis. Silica particles can in general also be obtained by precipitation of silica by means of silaffin polypeptides (silaffins). Silaffins are a family of proteins that are involved in silica biogenesis in cell walls of diatoms. These proteins have been shown to precipitate silica particles from a solution of silicic acid in vitro (C. C. Lechner and C. F. Becker, Mar. Drugs, 2015, 13, 5297-5333). Proteolytic processing of silaffins has revealed that the proteins comprise of repetitive units, one of which led to synthesis of a silaffin peptide R5 (sequence: SSKKSGSYSGSKGSKRRIL (SEQ ID NO: 1)) (C. C. Lechner and C. F. Becker, Mar. Drugs, 2015, 13, 5297-5333). Also the truncated peptide KRRIL (SEQ ID NO: 2) has been shown to precipitate silica with a random morphology (C. C. Lechner and C. F. W. Becker, J. Pept. Sci., 2014, 20, 152-158). The R5 peptide is highly efficient in precipitating monodisperse silica particles as a bio-inorganic matrix from a solution of silicic acid. The resulting SSPs are stable and are constituted by up to 50% organic matter in the form of the R5 peptide. Cargo molecules can be attached to the R5 peptide and the resulting product has been shown to form analogous silica particles but this time with cargo molecules as an integral part of the bio-inorganic matrix. The range of substances immobilized within SSPs in this manner spans from small molecules to full length proteins (C. C. Lechner and C. F. Becker, Biomater. Sci., 2015, 3, 288-297).

The proteins and polypeptides derived from diatoma that are known to be suitable for precipitating silica still bear significant drawbacks, in particular in the context of vaccination. First, such proteins and polypeptides may bear a non-negligible immunogenic effect by themselves, which evidently will disturb selective vaccination. Second, the given large-size proteins and polypeptides from diatoma are not easily accessible, in particular not by chemical peptide synthesis. Third, the given proteins and polypeptides from diatoma do not allow the individual adaptation of the morphology of the silica particles to the individually desired characteristics.

Accordingly, there is still the need for improved vaccine compositions which, on the one hand, show improved properties and enable the improved immune responses, which on the other hand, are concomitantly easily assessable by synthetic means under mild conditions enabling high and well-defined loading conditions.

Surprisingly, it has been found that short peptidic motifs can be used as pharmaceutically acceptable templates to efficiently form well-defined silica particles and enable efficient incorporation of antigens. This can be achieved by a single step allowing homogeneous loading efficiency. The peptidic motifs do not have to be removed prior to using the vaccine compositions obtained.

Accordingly, a first aspect of the present invention relates to a vaccine composition V comprising (or consisting of):
(A) a peptidic entity A comprising at least one sequence motif A1 of a sequence

(SEQ ID NO: 23)
RR-X^{1a}-X^{1b}-L,

wherein
X^{1a} is a hydrophobic amino acid moiety,
X^{1b} is a peptide bond or a hydrophobic amino acid moiety,
or a D-peptide analogue of the sequence SEQ ID NO: 23, wherein one or more of the L-amino acid moieties is/are replaced by the respective D-amino acid(s),
or a retro-inverso analogue of the sequence of SEQ ID NO: 23,
or a peptidomimetic analogue of the sequence of SEQ ID NO: 23;
(B) an antigen of interest B; and
(C) silica particles C embracing the peptidic entity A and the antigen of interest B; and
(D) optionally a pharmaceutically acceptable carrier.

It will be understood by a person skilled in the art that the present invention also refers to a composition comprising (or consisting of) the components A, B, C and optionally D in general. In other words, the composition does not necessarily have to be a vaccine composition V.

In a preferred embodiment, the peptidic entity A and the antigen of interest B together do not comprise more than 50 amino acid moieties, preferably not more than 40 amino acid moieties, in particular not more than 30 amino acid moieties.

Preferably, the antigen of interest B is a peptidic moiety and the peptidic entity A and the antigen of interest B together form a peptide strand AB that comprise more than 50 amino acid moieties, preferably not more than 40 amino acid moieties, in particular not more than 30 amino acid moieties.

In a preferred embodiment, the peptide strand AB comprises a peptide moiety having at least 80% sequence homology to SSKKSGSYSGSKGSKRRIL (SEQ ID NO: 1) or a peptidomimetic analogues of any thereof.

One example of a peptide strand AB is peptide 68:

(SEQ ID NO: 32)
(SIINFEKLCSSKKSGSYSGSKGSKRRIL.

In a preferred embodiment, sequence motif A1 is selected from the list consisting of

(SEQ ID NO: 4)
RRIL,
(SEQ ID NO: 5)
RRIIL,
(SEQ ID NO: 19)
LIRR,
(SEQ ID NO: 21)
LIIRR,
(SEQ ID NO: 33)
RRILL,
(SEQ ID NO: 34)
RRLL,
(D-analogue of SEQ ID NO: 4)
rril,
(D-analogue of SEQ ID NO: 5)
rriil,
(D-analogue of SEQ ID NO: 19)
lirr,
(D-analogue of SEQ ID NO: 21
liirr,
(D-analogue of SEQ ID NO: 33)
rrill,
(D-analogue of SEQ ID NO: 34)
rrll,

and
peptidomimetic analogues of any thereof

In a preferred embodiment, sequence motif A1 is selected from the list consisting of

(SEQ ID NO: 4)
RRIL,

rril (D-analogue of SEQ ID NO: 4), and
peptidomimetic analogues of any thereof

In a preferred embodiment, the vaccine composition V of the present invention comprises (or consists of):
(A) a peptidic entity A comprising:
   (A1) at least one first sequence motif A1 of a sequence

(SEQ ID NO: 23)
RR-X^{1a}-X^{1b}-L,

wherein
X^{1a} is a hydrophobic amino acid moiety,
X^{1b} is a peptide bond or a hydrophobic amino acid moiety,
or a D-peptide analogue of the sequence SEQ ID NO: 23, wherein one or more of the L-amino acid moieties is/are replaced by the respective D-amino acid(s),
or a retro-inverso analogue of the sequence of SEQ ID NO: 23,
or a peptidomimetic analogue of the sequence of SEQ ID NO: 23; and
(A2) at least one second sequence motif A2 of a sequence selected from the group consisting of

(SEQ ID NO: 23)
RR-X^{1a}-X^{1b}-L,
and
(SEQ ID NO: 24)
L-X^{1c}-X^{1d}-RR,

wherein
X^{1a} is a hydrophobic amino acid moiety,
X^{1b} is a peptide bond or a hydrophobic amino acid moiety,
X^{1c} is a hydrophobic amino acid moiety,
X^{1d} is a bond or a hydrophobic amino acid moiety,
or a D-peptide analogue of the sequence SEQ ID NO: 23 or 24, wherein one or more of the L-amino acid moieties is/are replaced by the respective D-amino acid(s),
or a retro-inverso analogue of the sequence of SEQ ID NO: 23 or 24,
or a peptidomimetic analogue of the sequence of SEQ ID NO: 23 or 24;
(B) an antigen of interest B; and
(C) silica particles C embracing the peptidic entity A and the antigen of interest B; and
(D) optionally a pharmaceutically acceptable carrier.

It will be understood by a person skilled in the art that the present invention also refers to a composition comprising (or consisting of) the components A, B, C and optionally D in general. In other words, the composition does not necessarily have to be a vaccine composition V.

More generally, the present invention also relates to a vaccine composition V comprising (or consisting of):
(A) a peptidic entity A comprising at least two sequence motifs each selected independently from another from the group consisting of

(SEQ ID NO: 23)
RR-X^{1a}-X^{1b}-L,
and
(SEQ ID NO: 24)
L-X^{1c}-X^{1d}-RR,

wherein
X^{1a} is a hydrophobic amino acid moiety,
X^{1b} is a peptide bond or a hydrophobic amino acid moiety,
X^{1c} is a hydrophobic amino acid moiety,
X^{1d} is a bond or a hydrophobic amino acid moiety,
or a D-peptide analogue of the sequence SEQ ID NO: 23 or 24, wherein one or more of the L-amino acid moieties is/are replaced by the respective D-amino acid(s),
or a retro-inverso analogue of the sequence of SEQ ID NO: 23 or 24,
or a peptidomimetic analogue of the sequence of SEQ ID NO: 23 or 24;
(B) an antigen of interest B; and
(C) silica particles C embracing the peptidic entity A and the antigen of interest B; and
(D) optionally a pharmaceutically acceptable carrier.

It will be understood that—according to general understanding by those skilled in the art—the terms “peptide”, “peptidic entity”, “polypeptide”, “protein”, “nucleic acid entities”, “oligo- or polysaccharide entity”, “lipid acid” and other biomolecules of course also include the salts thereof, in particular pharmaceutically acceptable salts. This of course also applies to all peptide sequences and amino acid (moieties) in the context of the present invention. If not otherwise indicated the peptides and amino acid moieties are preferably the L-peptides and L-amino acid moieties (or salts thereof).

As used herein, a vaccine composition V may be understood in the broadest sense as a composition that may provoke an immune response, in particular a secondary immune response and a cytotoxic immune response directed towards the antigen of interest B.

As used herein, the term “peptidic entity” may be understood in the broadest sense as any peptide or peptide residue a total of at least eight consecutive amino acid moieties and/or amino acid analogues comprising (i) at least one sequence motif A1 and at least one sequence motif A2 (arranged in any sequential order), or (ii) at least two sequence motifs A1 (arranged in any sequential order). It will be understood that in such peptide or peptide residue of at least eight consecutive amino acid moieties and/or amino acid analogues the amino acid moieties and/or amino acid analogues are typically directly or via a linker (e.g., an amino acid moiety or analogue thereof, a peptidic linker, such as, e.g., via a consecutive sequence of one, two, three four or more amino acid moieties or analogues thereof or any other linker) covalently conjugated with another, most preferably but not necessarily via one or more peptide bonds. Preferably, linkage of A1 and A2 (in any sequential order) may be via one amino acid moiety, in particular a lysine moiety. Then, linkage of A1 and A2 (in any sequential order) may be via the alpha or, in case of a lysine moiety, alternatively also the epsilon amino group. Typically but not necessarily, the linker between A1 and A2 (arranged in any sequential order) will have a length of less than 2 nm, preferably less than 1 nm. Alternatively, the linkage may also be non-covalent linkage.

Accordingly, a peptidic entity may be an independent molecular structure (i.e., an unbound peptide or peptide analogue (peptidomimetic)) or a peptide or peptidomimetic residue conjugated to another molecular structure. Exemplarily, the peptidic entity A may be any peptide comprising the sequence of SEQ ID NO: 3 or retro-inverso analogue or peptidomimetic thereof, but may also form part of a molecular structure embracing the sequence of SEQ ID NO: 3 and further comprising other molecular structures optionally including the antigen of interest B.

As used herein, the term “peptide bond” or “amide bond” may be understood interchangeably as any —CO—NH₂—, —CO—NR^{x}H— or —CO—NR^{x}R^{y}— group, wherein R^{x} and R^{y} are each independently from another any organic moiety preferably comprising not more than 20 carbon atoms, more preferably a C₁-C₄-alkyl or an amino acid side chain, preferably a naturally occurring side chain.

The term “peptidomimetic” may be understood in the broadest sense as any mimic of a peptide that has similar properties like a peptide, but typically bears higher (biological) stability. Examples for peptidomimetics in the sense of the present invention are such molecular structures partly or completely based on beta amino acid moieties, N-acetylated amino acid moieties (e.g., N-methylated amino acid moieties) and peptoids (i.e., poly-N-substituted glycinyl moieties). Preferably, if the sequence motifs A1 is a peptidomimetic, all amino acid moieties of the sequence motifs A1 are amino acid analogues of one type (e.g. all are on beta amino acid moieties, all are N-acetylated amino acid moieties or all are N-substituted glycinyl moieties). Likewise, if the sequence motifs A1 is a D-peptide analogue, all amino acid moieties of the sequence motifs A1 are D-amino acid moieties.

As used herein, the term “hydrophobic amino acid moiety” may be understood in the broadest sense as any hydrophobic amino acid moiety (naturally occurring or not, in particular having a molecular weight of not more than 500 Da). Preferably, a hydrophobic amino acid moiety is a natural hydrophobic amino acid moiety such as an amino acid moiety selected from the group consisting of isoleucine (Ile), leucine (Leu), valine (Val), alanine (Ala), phenylalanine (Phe), proline (Pro) and tryptophan (Trp). More preferably, a hydrophobic amino acid moiety is a natural aliphatic hydrophobic amino acid moiety, in particular an amino acid moiety selected from the group consisting of isoleucine, leucine and valine. Even more preferably, a hydrophobic amino acid moiety is selected from the group consisting of an isoleucine moiety and a valine moiety. Likewise, highly preferably, a hydrophobic amino acid moiety is selected from the group consisting of an isoleucine moiety and a leucine moiety. Particularly preferably, a hydrophobic amino acid moiety in the context of the present invention is an isoleucine moiety. In an alternative highly preferred embodiment, a hydrophobic amino acid moiety in the context of the present invention is a leucine moiety.

According to a preferred embodiment, the present invention relates to a vaccine composition V comprising (or consisting of):
(A) a peptidic entity A comprising:
   (A1) at least one first sequence motif A1 of a sequence

(SEQ ID NO: 23)
RR-X^{1a}-X^{1b}-L,

wherein
X^{1a} is selected from the group consisting of Ile, Leu and Val, preferably is Ile or Leu, in particular is Ile;
X^{1b} is a peptide bond or is selected from the group consisting of Ile, Leu and Val, preferably is Ile or Leu, in particular is Ile;
or a D-peptide analogue of the sequence SEQ ID NO: 23, wherein one or more of the L-amino acid moieties is/are replaced by the respective D-amino acid(s),
or a retro-inverso analogue of the sequence of SEQ ID NO: 23, or a peptidomimetic analogue of the sequence of SEQ ID NO: 23; and
(A2) at least one second sequence motif A2 of a sequence selected from the group consisting of

(SEQ ID NO: 23)
RR-X^{1a}-X^{1b}-L,
and
(SEQ ID NO: 24)
L-X^{1c}-X^{^{1d}}-RR,

wherein
X^{1a} is selected from the group consisting of Ile, Leu and Val, preferably is Ile or Leu, in particular is Ile;
X^{1b} is a peptide bond or is selected from the group consisting of Ile, Leu and Val, preferably is Ile or Leu, in particular is Ile;
X^{1c} is selected from the group consisting of Ile, Leu and Val, preferably is Ile or Leu, in particular is Ile;
X^{1d} is a bond or is selected from the group consisting of Ile, Leu and Val, preferably is Ile or Leu, in particular is Ile;
or a D-peptide analogue of the sequence SEQ ID NO: 23 or 24, wherein one or more of the L-amino acid moieties is/are replaced by the respective D-amino acid(s),
or a retro-inverso analogue of the sequence of SEQ ID NO: 23 or 24, or a peptidomimetic analogue of the sequence of SEQ ID NO: 23 or 24;
(B) an antigen of interest B, preferably wherein said antigen of interest is a peptide antigen of not more than 30 consecutive amino acids in length; and
(C) silica particles C embracing the peptidic entity A and the antigen of interest B; and
(D) optionally a pharmaceutically acceptable carrier.

It will be understood by a person skilled in the art that the present invention also refers to a composition comprising (or consisting of) the components A, B, C and optionally D in general. In other words, the composition does not necessarily have to be a vaccine composition V.

More generally, a preferred embodiment of the present invention relates to a vaccine composition V comprising (or consisting of):
(A) a peptidic entity A comprising at least two sequence motifs each selected independently from another from the group consisting of

(SEQ ID NO: 23)
RR-X^{1a}-X^{1b}-L,
and
(SEQ ID NO: 24)
L-X^{1c}-X^{1d}-RR,

wherein
X^{1a} is selected from the group consisting of Ile, Leu and Val, preferably is Ile or Leu, in particular is Ile;
X^{1b} is a peptide bond or is selected from the group consisting of Ile, Leu and Val, preferably is Ile or Leu, in particular is Ile;
X^{1c} is selected from the group consisting of Ile, Leu and Val, preferably is Ile or Leu, in particular is Ile;
X^{1d} is a bond or is selected from the group consisting of Ile, Leu and Val, preferably is Ile or Leu, in particular is Ile;
or a D-peptide analogue of the sequence SEQ ID NO: 23 or 24, wherein one or more of the L-amino acid moieties is/are replaced by the respective D-amino acid(s),
or a retro-inverso analogue of the sequence of SEQ ID NO: 23 or 24, or a peptidomimetic analogue of the sequence of SEQ ID NO: 23 or 24;
(B) an antigen of interest B; and
(C) silica particles C embracing the peptidic entity A and the antigen of interest B; and
(D) optionally a pharmaceutically acceptable carrier.

The peptidic structures (i.e., the peptidic entity as well as a peptidic antigen of interest B and all other peptidic residues) may be each independently from another be obtained by any means know for this purpose in the art. Preferably, the peptidic structures are obtained by solid phase peptide synthesis (SPPS) such as of Fmoc- or Boc-based SPPS. Alternatively, the peptidic structures may also be obtained by liquid phase peptide synthesis (LPPS) or, in the case of consisting of L-amino acid moieties, by means of biotechnology means such as heterologous expression in a genetically modified organism excluding human bodies such as, e.g., bacteria (e.g., E. coli), fungi (e.g., yeast), mammalian cells or mammalians excluding humans, insect cells or insects, plant cells or plants, etc. Accordingly, genetic manipulation of a host organism with the sequence comprising the peptidic motif A1 being genetically fused to a gene encoding for the peptidic motif A1 may be used. This may lead to the auto-encapsulation of the antigen of interest B inside the silica particle C during silica formation.

The term “retro-inverso analogue” will be unambiguously understood by those skilled in the art. In a retro-inverso analogue, the respective sequence is reversed and D-amino acid moieties are used instead of L-amino acid moieties.

It will be understood that also a combination of two or more of these methods may be used.

In SPPS (or also LPPS), the synthesis typically bases on the stepwise coupling of amino acid moieties bearing protected side chains (orthogonal protecting groups). Typically, during synthesis, the peptide strand grows from the C-terminus to the N-terminus. However, there are alternative methods wherein the peptide strand grows from the N-terminus to the C-terminus. Nowadays, the most common methods base on at least two different types of protecting groups that are cleavable under at least two different conditions, such as, e.g., the fluorenyl-9-methoxycarbonyl/tert-butanyl-(Fmoc/tBu) protecting group scheme (Sheppard Tactics) or the tert-butoxycarbonyl/benzyl-(Boc/Bzl) protecting group scheme (Merrifield Tactics). Alternatively or additionally, the peptidic structures (i.e., the peptidic entity as well as a peptidic antigen of interest B and all other peptidic residues) may be also provided by conjugating two or more peptide strand(s) with another by any conjugation method known in the art such as, e.g., Native Chemical Ligation (NCL), Click Chemistry, Maleimide-Thiol Conjugation, enzymatic conjugation, biochemical protein ligation and/or soluble handling conjugation. Alternatively, the peptidic structure may be obtained from a biotechnological method. The peptidic structure may further be extracted by any means known in the art. Additionally, the peptidic structures may be purified by any means known in the art, such as, e.g., one or more chromatographic method(s), one or more filtration method(s), one or more electrophoretic method(s), one or more precipitation-based method(s), one or more dialysis method(s) or a combination of two or more thereof. The natural source may be any biological material such as, e.g., bacterial material, plant material, animal material or fungal material, such as e.g. tissue, liquids or secretion(s). In particular a peptidic structure obtained from a biotechnological method or a natural source may further be purified and/or modified by chemical means known in the art.

As used herein, the terms “solid support” in the context of peptide synthesis may be understood interchangeably in the broadest sense as any solid matrix known for peptide synthesis in the art. Typically the solid support is a plastic bead. The solid support may be, but may not be limited to, chloromethyl resin (Merrifield resin), 4-benzyloxybenzyl alcohol resin (Wang resin), (2,4-dimethoxy)benzhydrylamine resin (Rink amide resin), 2,4-dialkoxybenzyl resin (super acid-sensitive resin, SASRIN®), 2-chlorotrityl resin, alpha-chlorotritylchloride resin (Barlos resin), benzhydrylamine resin (BHA resin), chloromethyl resin, hydroxymethylbenzoic acid resin (HMBA resin), 4-hydroxymethyl-3-methoxyphenoxybutyric acid resin (HMPB resin), hydroxycrotonoyl aminomethyl resin (HYCRAM resin), MBHA resin, oxime resin, 4-(hydroxymethyl)phenylacetamidomethyl resin (PAM resin) and/or a resin with special cleavable linkers (e.g., photolabile linkers or safety-catch linkers).

Solid phase peptide synthesis (SPPS) methods typically base on the use of orthogonal protecting groups cleavable under special conditions. A protecting group may be any protecting group known in the art such as, e.g., an amino-protecting group of the urethane type (e.g., benzyloxycarbonyl (Z), 4-methoxybenzyloxycarbonyl (Z(OMe)), 2-nitrobenzyloxycarbonyl (Z(2-NO₂)), 4-nitrobenzyloxycarbonyl (Z(NO₂)), chlorobenzyloxycarbonyl (Z(Cl), Z(2-Cl), Z(3-Cl), Z(2,4-Cl)), 3,5-dimethoxybenzyloxycarbonyl (Z(3,5-OMe), alpha,alpha-dimethyl-3,5-dimethoxybenzyloxycarbonyl (Ddz), 6-nitroveratryloxycarbonyl (Nvoc), 4-(phenyldiazenyl)-benzyloxycarbonyl (Pz), a-methyl-2,4,5-trimethylbenzyloxycarbonyl (Bic), 2-(biphenyl-4-yl)-2-propoxycarbonyl (Bpoc), (4-phenylazophenyl)-isopropoxycarbonyl (Azoc), isonicotinyloxycarbonyl (iNoc), tert-butoxycarbonyl (Boc), 2-cyano-tert-butoxycarbonyl (Cyoc), 2,2,2-trichloro-tert-butoxycarbonyl (Tcboc), adamantyl-1-oxycarbonyl (Adoc), 1-(1-adamantyl)-1-methoxycarbonyl (Adpoc), isobornyloxycarbonyl (Iboc), fluorenyl-9-methoxycarbonyl (Fmoc), (2-nirofluoren-9-yl)methoxycarbonyl (Fmoc(NO₂)), 2-(4-toluenesulfonyl)-ethoxycarbonyl (Tsoc), methylsulfonylethoxycarbonyl (Msc), 2-(4-nitrophenylsulfonyl)ethoxycarbonyl (Nsc), 2-(tert-butylsulfonyl)-2-propenyloxycarbonyl (Bspoc), 1,1-dioxobenzo[b]-thien-2-ylmethoxycarbonyl (Bsmoc), 2-/methylsulfonyl)-3-phenyl-2-propenyloxycarbonyl (Mspoc), allyloxycarbonyl (Aloc), 2-(trimethylsilyl)-ethoxycarbonyl (Teoc), triisopropylsilylethoxycarbonyl (Tipseoc), piperidinyloxycarbonyl (Pipoc), cyclopententyloxycarbonyl (Poc), 3-nitro-1,5-dioxaspiro[5.5]undec-3-ylmethoxycarbonyl (PTnm), 2-ethynyl-2-propyl-oxycarbonyl (Epoc)), a carboxy-protecting group of the ester type (e.g., methyl (Me), ethyl (Et), benzyl (Bzl), 4-nitrobenzyl (Nbz), 4-methoxybenzyl (Mob), 2,4-di methoxybenzyl (2,4-Dmb), o-chlorotrityl (Trt(2-Cl), pyrimidyl-4-methyl(4-picolyl (Pic), 2-toluene-4-sulfonyl)-ethyl (Tse), phenacyl (Pac), 4-methoxyphenacyl (Pac(OMe), diphenylmethyl (Dpm), tert-butyl (tBu), cyclohexyl (Cy), 1-adamantyl (1-Ada), 2-adamantyl (2-Ada), dicyclopropylmethyl (Dcpm), 9-phenylfluoren-9-yl (Pf), 9-fluorenylmethyl (Fm), 2-trimethylsilylethyl (TMSE), 2-phenyl-trimethyl-silyl (PTMSE), allay (Al), 4-{N-[1-(4,4-dimethyl-2,6-dioxocyclohexanylidene)-3-methylbutyl]-amino}benzyl (Dmab)), a thiol-protecting group (e.g., benzyl (Bzl), 4-methylbenzyl (Bzl(4-Me)), 4-methoxybenzyl (Mob), 2,4,6-trimethoxybenzyl (Tmb), diphenylmethyl (Dpm), trityl (Trt), tert-butyl (tBu), acetamidomethyl (Acm), trimethylacetamidomethyl (Tacm), 9-fluorophenylmethyl (Fm), tert-butylsulfanyl (StBu), 3-nitro-2-pyridylsulfanyl (Npys), allyloxycrbanylaminomethyl (Alocam), 9H-xanthen-9-yl (Xan)), an imidazole protecting group (e.g., benzyl (Bzl), 2,4-dinitrophenyl (Dnp), benzyloxycarbonyl (Bom), adamantly-1-oxycarbonyl (Adoc), triphenylmethyl (Trt), diphenylmethyl (Dpm), pyridyldiphenylmethyl (Pdpm), 4-toluenesulfonyl (Tosyl, Tos), 4-methoxybenzenesulfonyl (Mbs), tert-butoxymethyl (Burn), allyl (Al), allyloxymethyl (Alom)), or a hydroxyl-protecting group (e.g., benzyl (Bzl), 2,6-dichlorobenzyl (Dcb), diphenylmethyl (Dbm), cyclohexyl (Cy), 2-bromobenzyloxycarbonyl (Z(2-Br)), tert-butyl (tBu), 1-benzyloxycarbonyl-amino-2,2,2-trifluoroethyl (Zte), methylthiomethyl (Mtm), allyl (Al), allylcarbonyl (Aloc)).

In a preferred embodiment, at least one X^{1a} is an isoleucine or a leucine moiety, X^{1c} is an isoleucine or a leucine moiety, at least one X^{1b} is a peptide bond or isoleucine or a leucine moiety, and/or X^{1c} is a peptide bond or a or leucine isoleucine moiety.

In a more preferred embodiment, at least one X^{1a} is an isoleucine moiety, X^{1c} is an isoleucine or a leucine moiety, at least one X^{1b} is a peptide bond or isoleucine or a leucine moiety, and/or X^{1c} is a peptide bond or a or leucine isoleucine moiety.

In a more preferred embodiment, at least one X^{1a} is an isoleucine or a leucine moiety, X^{1c} is an isoleucine moiety, at least one X^{1b} is a peptide bond or isoleucine or a leucine moiety, and/or X^{1c} is a peptide bond or a or leucine isoleucine moiety.

In a more preferred embodiment, at least one X^{1a} is an isoleucine moiety, X^{1c} is an isoleucine moiety, at least one X^{1b} is a peptide bond or isoleucine moiety, and/or X^{1c} is a peptide bond or isoleucine moiety.

Accordingly, the residues X^{1a}, X^{1b}, X^{1c} and X^{1d} may preferably have the following meanings:

X^{1a}=any hydrophobic amino acid moiety, X^{1c}=isoleucine moiety, X^{1b}=peptide bond, and X^{1c}=peptide bond;
X^{1a}=isoleucine moiety, X^{1c}=any hydrophobic amino acid moiety, X^{1b}=peptide bond, and X^{1c}=peptide bond;
X^{1a}=isoleucine moiety, X^{1c}=any hydrophobic amino acid moiety, X^{1b}=isoleucine moiety, and X^{1c}=peptide bond;
X^{1a}=any hydrophobic amino acid moiety, X^{1c}=isoleucine moiety, X^{1b}=isoleucine moiety, and X^{1c}=peptide bond;
X^{1a}=isoleucine moiety, X^{1c}=isoleucine moiety, X^{1b}=X^{1a}=any hydrophobic amino acid moiety, X^{1c}=isoleucine moiety, X^{1b}=isoleucine moiety, and X^{1c}=peptide bond;
and X^{1c}=peptide bond;
X^{1a}=any hydrophobic amino acid moiety, X^{1c}=isoleucine moiety, X^{1b}=peptide bond, and X^{1c}=isoleucine moiety;
X^{1a}=isoleucine moiety, X^{1c}=isoleucine moiety, X^{1b}=peptide bond, and X^{1c}=any hydrophobic amino acid moiety;
X^{1a}=isoleucine moiety, X^{1c}=any hydrophobic amino acid moiety, X^{1b}=peptide bond, and X^{1c}=isoleucine moiety;
X^{1a}=any hydrophobic amino acid moiety, X^{1c}=isoleucine moiety, X^{1b}=isoleucine moiety, and X^{1c}=isoleucine moiety;
X^{1a}=isoleucine moiety, X^{1c}=any hydrophobic amino acid moiety, X^{1b}=isoleucine moiety, and X^{1c}=isoleucine moiety;
X^{1a}=isoleucine moiety, X^{1c}=isoleucine moiety, X^{1b}=any hydrophobic amino acid moiety, and X^{1c}=isoleucine moiety; or
X^{1a}=isoleucine moiety, X^{1c}=isoleucine moiety, X^{1b}=isoleucine moiety, and X^{1c}=any hydrophobic amino acid moiety.

Accordingly, particularly preferably, the residues X^{1a}, X^{1b}, X^{1c} and X^{1d} have the following meanings:

X^{1a}=isoleucine moiety, X^{1c}=isoleucine moiety, X^{1b}=peptide bond, and X^{1c}=peptide bond;
X^{1a}=isoleucine moiety, X^{1c}=isoleucine moiety, X^{1b}=isoleucine moiety, and X^{1c}=peptide bond;
X^{1a}=isoleucine moiety, X^{1c}=isoleucine moiety, X^{1b}=peptide bond, and X^{1c}=isoleucine moiety; or
X^{1a}=isoleucine moiety, X^{1c}=isoleucine moiety, X^{1b}=isoleucine moiety, and X^{1c}=isoleucine moiety.

It will be understood that these preferred definitions also refer to the respective corresponding D-peptides.

As indicated above, the peptidic entity A comprises one or more sequence motifs A1 of the sequence SEQ ID NO: 23 or a D-peptide analogue thereof, wherein one or more of the L-amino acid moieties is/are replaced by the respective D-amino acid(s).

Preferably, the peptidic entity A comprises one or more sequence motifs A1 of the sequence SEQ ID NO: 23 or a D-peptide analogue thereof, wherein all L-amino acid moieties are replaced by the respective D-amino acid moieties. Accordingly, the peptidic entity A preferably comprises one or more sequence motifs A1 of at least one of the two sequences a. and/or b.

a.

(SEQ ID NO: 23)
RR-X^{1a}-X^{1b}-L,

wherein
X^{1a} is a hydrophobic amino acid moiety,
X^{1b} is a peptide bond or a hydrophobic amino acid moiety, or
   b.

(D-equivalent of SEQ ID NO: 23)
rr-x^{1a}-x^{1b}-l

X^{1a} is a hydrophobic amino acid moiety,
X^{1b} is a peptide bond or a hydrophobic D-amino acid moiety, or

Preferably, the peptidic entity A comprises one or more sequence motifs A2 of the sequence SEQ ID NO: 24 or a D-peptide analogue thereof, wherein all L-amino acid moieties are replaced by the respective D-amino acid moieties. Accordingly, the peptidic entity A preferably comprises one or more sequence motifs A1 of at least one of the two sequences a. and/or b.

a.

(SEQ ID NO: 23)
RR-X^{1a}-X^{1b}-L,
or
(SEQ ID NO: 24)
L-X^{1c}-X^{1d}-RR,

wherein
X^{1a} is a hydrophobic amino acid moiety,
X^{1b} is a peptide bond or a hydrophobic amino acid moiety, or
X^{1c} is a hydrophobic amino acid moiety,
X^{1d} is a peptide bond or a hydrophobic amino acid moiety; or
   b.

(D-equivalent of SEQ ID NO: 23)
rr-x^{1a}-x^{1b}-l,
or
(D-equivalent of SEQ ID NO: 24)
l-x^{1a}-x^{1b}-rr

x^{1a} is a hydrophobic amino acid moiety,
x^{1b} is a peptide bond or a hydrophobic D-amino acid moiety, or
x^{1c} is a hydrophobic amino acid moiety,
x^{1d} is a peptide bond or a hydrophobic D-amino acid moiety; or

As indicated above, the peptidic entity A comprises one or more sequence motifs A1 of the sequence SEQ ID NO: 3 or a D-peptide analogue thereof, wherein one or more of the L-amino acid moieties is/are replaced by the respective D-amino acid(s).

Preferably, the peptidic entity A comprises one or more sequence motifs A1 of the sequence SEQ ID NO: 3 or a D-peptide analogue thereof, wherein all L-amino acid moieties are replaced by the respective D-amino acid moieties. Accordingly, the peptidic entity A preferably comprises one or more sequence motifs A1 of at least one of the two sequences a. and/or b.

a.

(SEQ ID NO: 3)
RRI-X¹-L,

wherein X¹ is either a peptide bond (between the preceding isoleucine moiety (also designatable as isoleucinyl moiety, isoleucine moiety residue, isoleucinyl residue, Ile, or the like) and the subsequent leucine moiety) or an L-isoleucine moiety; or
b. rri-x¹-l
   wherein x¹ is either a peptide bond (between the preceding isoleucine moiety and the subsequent leucine moiety) or an D-isoleucine moiety.

More in detail, the peptidic entity A preferably comprises one or more sequence motifs A1 selected from the group consisting of:

(SEQ ID NO: 4)
RRIL
(SEQ ID NO: 5)
RRIIL

rril (D-analogue of SEQ ID NO: 4); and
rriil (D-analogue of SEQ ID NO: 5).

In a preferred embodiment, the vaccine composition V comprises (or consists of)
(A) a peptidic entity A comprising (or consisting of) one or more sequence motifs A1 of the sequence
   RRI-X¹-L (SEQ ID NO: 3),
   wherein X¹ is either a peptide bond or an isoleucine moiety,
   or a D-peptide analogue thereof, wherein one or more of the L-amino acid moieties is/are replaced by the respective D-amino acid(s),
   or a retro-inverso analogue of the sequence of SEQ ID NO: 3,
   or a peptidomimetic analogue of the sequence of SEQ ID NO: 3;
(B) an antigen of interest B; and
(C) silica particles C embracing the peptidic entity A and the antigen of interest B; and
(D) optionally a pharmaceutically acceptable carrier.

As used throughout the present invention, the designation of the amino acid moieties and chemical structures follows the common designations. Accordingly, the peptide sequences are depicted beginning with the N-terminal amino acid moiety to the C-terminal amino acid in reading order. The L-amino acid moieties are indicated by capital letters in the one letter code. As far as the three letter code is used (e.g., in the sequence listing enclosed herewith), a typical combination of one capital and two small letters (minuscule) is used. The respective abbreviations are well-known to those skilled in the art and can be obtained by any standard textbook in the field of biochemistry or peptide chemistry. The respective D-amino acid moieties are indicated by small letters (minuscule) n the one letter code.

The abbreviation “(εK)” indicates an L-lysine residue that is bound via its epsilon amino group to the preceding amino acid moiety in the sequence. Accordingly, the abbreviation “(εk)” indicates a D-lysine residue that is bound via its epsilon amino group to the preceding amino acid moiety in the sequence. In some cases, this epsilon amino group conjugation is also depicted by expressly depicting the chemical structure of this amino acid moiety (cf., FIG. 1).

Preferably, the vaccine compositions V is a pharmaceutical composition comprising one or more pharmaceutically acceptable carriers.

As used herein, the terms “pharmaceutically acceptable carrier”, “pharmaceutically acceptable excipient”, “carrier” and “excipient” may be understood interchangeably in the broadest sense as any substance that may support the pharmacological acceptance of the vaccine composition V.

Preferred vaccine compositions V prepared for final administration enable routes of administration which circumvent the first pass effect. More preferably, the pharmaceutical composition is prepared to be suitable for administration by injection into the patient (e.g., suitable for administration routes selected from the group consisting of intravenous (i.v.), intraarterial (i.a.), intraperitoneal (i.p.), intramuscular (i.m.), and subcutaneous (s.c.) injection). Alternatively or additionally, the pharmaceutical composition may also be suitable for other routes of administration such as, e.g., nasal or transdermal administration.

The pharmaceutical composition ready to use preferably is a liquid formulation, in particular an injection portion. The storage form may also be liquid, but may also be a dried form (e.g. a powder such as a powder comprising dried or freeze-dried silica particles C embracing the peptidic entity A and the antigen of interest B) or may be a paste or syrup or the like. Optionally, a dried form, paste or syrup may be dissolved or emulsified prior to being administered to the patient.

A pharmaceutically acceptable carrier may exemplarily be selected from the list consisting of an aqueous buffer, saline, water, dimethyl sulfoxide (DMSO), ethanol, vegetable oil, paraffin oil or combinations of two or more thereof. Furthermore, the pharmaceutically acceptable carrier may optionally contain one or more detergent(s), one or more foaming agent(s) (e.g., sodium lauryl sulfate (SLS), sodium doceyl sulfate (SDS)), one or more coloring agent(s) (e.g., food coloring), one or more vitamin(s), one or more salt(s) (e.g., sodium, potassium, calcium, zinc salts), one or more humectant(s) (e.g., sorbitol, glycerol, mannitol, propylenglycol, polydextrose), one or more enzyme(s), one or more preserving agent(s) (e.g., benzoic acid, methylparabene, one or more antioxidant(s), one or more herbal and plant extract(s), one or more stabilizing agent(s), one or more chelating agents (e.g., ethylenediaminetetraacetic acid (EDTA), and/or one or more uptake mediator(s) (e.g., polyethylene imine (PEI), a cell-penetrating peptide (CPP), a protein transduction domain (PTD), an antimicrobial peptide, etc.).

The present invention also relates to a dosage unit of the pharmaceutical composition of the present invention. Exemplarily, the present invention may refer to a single dose container or to a multiple dosage form.

Preferably, the one or more sequence motifs A1 is/are not as such immunogenic. In other words, a sequence motifs A1 is preferably not serving as antigen by itself.

In a preferred embodiment, the peptidic entity A may comprise one, two, three, four or more than four sequence motifs A1. In a preferred embodiment, the peptidic entity A may comprise one, two, three, four or more than four sequence motifs A2. In a more preferred embodiment, the peptidic entity A comprises one or two sequence motif(s) A1 and one or two sequence motif(s) A2. In a particularly preferred embodiment, the peptidic entity A comprises a single (i.e., not more than one) sequence motif A1 and a single i.e., not more than one) sequence motif A2. The sequence motifs A1 and A2 may be the same or may be different. These may be directly consecutively adjacent to another or may be spatially/sequentially separated by a spacer of one or more amino acid moieties.

In a preferred embodiment, the peptidic entity A comprises (or consists of) a sequence motif A12 selected from the group consisting of:

(SEQ ID NO: 25)
RR-X^{1a}-X^{1b}-L-X²-RR-X^{3a}-X^{3b}-L
(SEQ ID NO: 26)
L-X^{1a}-X^{1b}-RR-X²-RR-X^{3a}-X^{3b}-L,
and
(SEQ ID NO: 27)
RR-X^{3a}-X^{3b}-L-X²-L-X^{1a}-X^{1b}-RR,

wherein
X^{1a} and X^{1b} each independently from another have the same meaning as defined as above,
X^{3a} and X^{3b} each independently from another have the same meaning as X^{1a} and X^{1b}, and
X² is linker moiety of up to 100 carbon atoms,
or a D-peptide analogue of the sequence SEQ ID NO: 25, 26 or 27, wherein one or more of the L-amino acid moieties is/are replaced by the respective D-amino acid(s),
or a retro-inverso analogue of the sequence of SEQ ID NO: 25, 26 or 27,
or a peptidomimetic analogue of the sequence of SEQ ID NO: 25, 26 or 27.

In a preferred embodiment, the peptidic entity A comprises (or consists of) a sequence motif A12 selected from the group consisting of:

(SEQ ID NO: 25)
RR-X^{1a}-X^{1b}-L-X²-RR-X^{3a}-X^{3b}-L
(SEQ ID NO: 26)
L-X^{1a}-X^{1b}-RR-X²-RR-X^{3a}-X^{3b}-L,
and
(SEQ ID NO: 27)
RR-X^{3a}-X^{3b}-L-X²-L-X^{1a}-X^{1b}-RR,

wherein
X^{1a} and X^{1b} each independently from another have the same meaning as defined as above,
X³² and X^{3b} each independently from another have the same meaning as X^{1a} and X^{1b}, and
X² is a peptide moiety of not more than five consecutive amino acid moieties, preferably wherein X² comprises or consists of K or εK optionally substituted by a sequence of four or five consecutive amino acid moieties conjugated to the second amino group of the lysine moiety,
or a D-peptide analogue of the sequence SEQ ID NO: 25, 26 or 27, wherein one or more of the L-amino acid moieties is/are replaced by the respective D-amino acid(s),
or a retro-inverso analogue of the sequence of SEQ ID NO: 25, 26 or 27,
or a peptidomimetic analogue of the sequence of SEQ ID NO: 25, 26 or 27.

Preferably, X² is a peptide moiety of not more than four consecutive amino acid moieties, more preferably of not more than three consecutive amino acid moieties, even more preferably of not more than two consecutive amino acid moieties, in particular of a single amino acid moiety. Particularly preferably, X² is K or εK. Optionally, the K or εK. is substituted by a sequence of four or five consecutive amino acid moieties conjugated to the second amino group of the lysine moiety, in particular by a sequence of SEQ ID NO: 23 or 24 or particularly preferable embodiments thereof.

In an alternative preferred embodiment, in any of the sequences SEQ ID Nos: 25, 26 or 27, X² is an alternative linker moiety of up to 100 carbon atoms. In a preferred embodiment, X² is a polyethylene glycol (PEG) linker with 1 to 20 consecutive PEG units such as, e.g., a (PEG)₁, (PEG)₂, (PEG)₃, (PEG)₄, or (PEG)₅. It will be understood that throughout the present invention, alternative linker moiety X² (such as, e.g., a PEG linker moiety) preferably bears an amino group moiety at one terminus conjugated to a carboxylic acid moiety of an amino acid moiety and an carboxyl moiety at the other terminus conjugated to an amino moiety acid moiety of an amino acid moiety. Thus, in a preferred embodiment, the structure of a PEG linker may be selected from the group consisting of the following:

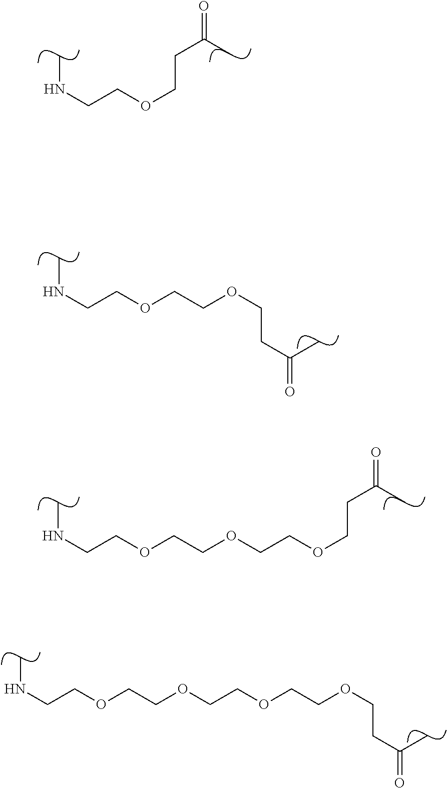

Herein, the wavy line (˜) indicates the binding site to the amino acid motifs.

Alternatively, the linker moiety X² may also be an carbohydrate linker of the formula —NH—(CH₂)ₙ—CO—, wherein n=1, 2, 3, 4, 5, 6, 7, 8, 9, or 10.

In a D-peptide analogue of the sequence SEQ ID NO: 25, 26 or 27, preferably one or both sequence motifs A1 and/or A2, i.e., RR-X^{1a}-X^{1b}-L (SEQ ID NO: 23) and/or L-X^{1c}-X^{1d}-RR (SEQ ID NO: 24), is/are replaced by D-amino acid moieties as a whole, i.e., rr-x^{1a}-x^{1b}-l (D-analogue of SEQ ID NO: 23) and/or 1-x^{1c}-x^{id}-rr (D-analogue of SEQ ID NO: 24), respectively.

In a further preferred embodiment, the peptidic entity A comprises (or consists of) the sequence motif A12:

(SEQ ID NO: 15)
RRI-X¹-L-X²-RRI-X³-L
or
(SEQ ID NO: 20)
L-X¹-IRR-X²-RRI-X³-L,

wherein each X¹ and X³ are independently from another either a peptide bond, a leucine moiety or an isoleucine moiety, and X² is a linker moiety of up to 100 carbon atoms, preferably wherein said X² is a peptide moiety optionally substituted by a further sequence motif A3 which is equal to sequence motif A2,
or a D-peptide analogue thereof, wherein one or more of the L-amino acid moieties is/are replaced by the respective D-amino acid(s).

In a further preferred embodiment, the peptidic entity A comprises (or consists of) the sequence motif A12:

(SEQ ID NO: 15)
RRI-X¹-L-X²-RRI-X³-L
or
(SEQ ID NO: 20)
L-X¹-IRR-X²-RRI-X³-L,

wherein each X¹ and X³ are independently from another either a peptide bond or an isoleucine moiety, and X² is a peptide moiety optionally substituted by a further sequence motif A3 which is equal to sequence motif A2, preferably wherein said X2 is a peptide moiety is one amino acid moiety optionally substituted by a further sequence motif A3 which is equal to sequence motif A2,
or a D-peptide analogue thereof, wherein one or more of the L-amino acid moieties is/are replaced by the respective D-amino acid(s).

Preferably, in such D-peptide analogue at least one motif RRIL (SEQ ID NO: 4) is (as a whole) replaced by its D-peptide analogue rril and/or at least one motif RRIIL (SEQ ID NO: 5) is (as a whole) replaced by its D-peptide analogue rriil, and optionally the lysine moiety K (also designatable as lysinyl moiety, lysine residue, lysinyl residue, Lys, or the like) is replaced by its D-amino acid analogue k or the lysine moiety (εK) is replaced by its D-amino acid analogue (εk).

Preferably, X² in the sequence motif A12 of SEQ ID NO: 15 or 20 is a lysine moiety. This may be conjugated with the preceding leucine moiety (also designatable as leucinyl moiety, leucine residue, leucinyl residue, Leu, or the like) by its alpha or its epsilon amino group. Accordingly, X² may be K or (εK).

In the sequence motif A12 of SEQ ID NO: 15 or 20, the following definitions are preferably selected from the group consisting of:

X¹=a peptide bond, X²=any amino acid moiety and X³=a peptide bond;
X¹=isoleucine moiety, X²=any amino acid moiety and X³=a peptide bond;
X¹=a peptide bond, X²=any amino acid moiety and X³=a isoleucine moiety;
X¹=isoleucine moiety, X²=any amino acid moiety and X³=a isoleucine moiety;
X¹=a peptide bond, X²=alpha lysine moiety and X³=a peptide bond;
X¹=isoleucine moiety, X²=alpha lysine moiety and X³=a peptide bond;
X¹=a peptide bond, X²=alpha lysine moiety and X³=a isoleucine moiety;
X¹=isoleucine moiety, X²=alpha lysine moiety and X³=a isoleucine moiety;
X¹=a peptide bond, X²=epsilon lysine moiety and X³=a peptide bond;
X¹=isoleucine moiety, X²=epsilon lysine moiety and X³=a peptide bond;
X¹=a peptide bond, X²=epsilon lysine moiety and X³=a isoleucine moiety;
X¹=isoleucine moiety, X²=epsilon lysine moiety and X³=a isoleucine moiety.
X¹=a peptide bond, X²=any amino acid moiety and X³=a peptide bond;
X¹=leucine moiety, X²=any amino acid moiety and X³=a peptide bond;
X¹=a peptide bond, X²=any amino acid moiety and X³=a isoleucine moiety;
X¹=leucine moiety, X²=any amino acid moiety and X³=a isoleucine moiety;
X¹=a peptide bond, X²=alpha lysine moiety and X³=a peptide bond;
X¹=leucine moiety, X²=alpha lysine moiety and X³=a peptide bond;
X¹=a peptide bond, X²=alpha lysine moiety and X³=a isoleucine moiety;
X¹=leucine moiety, X²=alpha lysine moiety and X³=a isoleucine moiety;
X¹=a peptide bond, X²=epsilon lysine moiety and X³=a peptide bond;
X¹=leucine moiety, X²=epsilon lysine moiety and X³=a peptide bond;
X¹=a peptide bond, X²=epsilon lysine moiety and X³=a isoleucine moiety;
X¹=leucine moiety, X²=epsilon lysine moiety and X³=a isoleucine moiety.
X¹=a peptide bond, X²=any amino acid moiety and X³=a peptide bond;
X¹=isoleucine moiety, X²=any amino acid moiety and X³=a peptide bond;
X¹=a peptide bond, X²=any amino acid moiety and X³=a leucine moiety;
X¹=isoleucine moiety, X²=any amino acid moiety and X³=a leucine moiety;
X¹=a peptide bond, X²=alpha lysine moiety and X³=a peptide bond;
X¹=isoleucine moiety, X²=alpha lysine moiety and X³=a peptide bond;
X¹=a peptide bond, X²=alpha lysine moiety and X³=a leucine moiety;
X¹=isoleucine moiety, X²=alpha lysine moiety and X³=a leucine moiety;
X¹=a peptide bond, X²=epsilon lysine moiety and X³=a peptide bond;
X¹=isoleucine moiety, X²=epsilon lysine moiety and X³=a peptide bond;
X¹=a peptide bond, X²=epsilon lysine moiety and X³=a leucine moiety;
X¹=isoleucine moiety, X²=epsilon lysine moiety and X³=a leucine moiety.
X¹=a peptide bond, X²=any amino acid moiety and X³=a peptide bond;
X¹=leucine moiety, X²=any amino acid moiety and X³=a peptide bond;
X¹=a peptide bond, X²=any amino acid moiety and X³=a leucine moiety;
X¹=leucine moiety, X²=any amino acid moiety and X³=a leucine moiety;
X¹=a peptide bond, X²=alpha lysine moiety and X³=a peptide bond;
X¹=leucine moiety, X²=alpha lysine moiety and X³=a peptide bond;
X¹=a peptide bond, X²=alpha lysine moiety and X³=a leucine moiety;
X¹=leucine moiety, X²=alpha lysine moiety and X³=a leucine moiety;
X¹=a peptide bond, X²=epsilon lysine moiety and X³=a peptide bond;
X¹=leucine moiety, X²=epsilon lysine moiety and X³=a peptide bond;
X¹=a peptide bond, X²=epsilon lysine moiety and X³=a leucine moiety; and
X¹=leucine moiety, X²=epsilon lysine moiety and X³=a leucine moiety.

Preferably, in the sequence motif A12 of SEQ ID NO: 15 or 20, the following definitions are preferably selected from the group consisting of:

X¹=a peptide bond, X²=any amino acid moiety and X³=a peptide bond;
X¹=isoleucine moiety, X²=any amino acid moiety and X³=a peptide bond;
X¹=a peptide bond, X²=any amino acid moiety and X³=a isoleucine moiety;
X¹=isoleucine moiety, X²=any amino acid moiety and X³=a isoleucine moiety;
X¹=a peptide bond, X²=alpha lysine moiety and X³=a peptide bond;
X¹=isoleucine moiety, X²=alpha lysine moiety and X³=a peptide bond;
X¹=a peptide bond, X²=alpha lysine moiety and X³=a isoleucine moiety;
X¹=isoleucine moiety, X²=alpha lysine moiety and X³=a isoleucine moiety;
X¹=a peptide bond, X²=epsilon lysine moiety and X³=a peptide bond;
X¹=isoleucine moiety, X²=epsilon lysine moiety and X³=a peptide bond;
X¹=a peptide bond, X²=epsilon lysine moiety and X³=a isoleucine moiety; and
X¹=isoleucine moiety, X²=epsilon lysine moiety and X³=a isoleucine moiety.

It will be understood that these preferred definitions also refer to the respective corresponding D-peptides.

In a preferred embodiment, the sequence motif A12 is selected from the group consisting of:

(SEQ ID NO: 6)
RRILKRRIL;
(SEQ ID NO: 7)
RRIL(εK)RRIL;
(SEQ ID NO: 8)
RRIIL(εK)RRIL;
(SEQ ID NO: 9)
RRILKRRIIL;
(SEQ ID NO: 10)
RRIILKRRIL;
(SEQ ID NO: 11)
RRIILKRRIIL;
(SEQ ID NO: 12)
RRIL(εK)RRIIL;
(SEQ ID NO: 13)
RRIIL(εK)RRIIL;
(SEQ ID NO: 16)
LIRRKRRIL;
(SEQ ID NO: 17)
LIRR(εK)RRIL;
(SEQ ID NO: 18)
RRILKLIIRR;
(SEQ ID NO: 29)
RRILL(εK)RRIL;
(SEQ ID NO: 30)
RRIIL(εK)RRLL;
(SEQ ID NO: 31)
RRIL(PEG)₃RRIL;
(SEQ ID NO: 28)
LIRRK(X⁴)RRIL,

wherein X⁴ is a sequence of four or five consecutive amino acid moieties conjugated to the epsilon amino group of the lysine moiety, preferably is a sequence of SEQ ID NO: 23 or SEQ ID NO: 24;
and a D-peptide analogue of any of these sequences of SEQ ID NO: 6-13, 16-18 or 28, wherein in said D-peptide analogue at least one motif RRIL (SEQ ID NO: 4) is replaced by its D-peptide analogue rril, and/or at least one motif RRIIL (SEQ ID NO: 5) is replaced by its D-peptide analogue rriil, and/or at least one motif LIRR (SEQ ID NO: 19) is replaced by its D-peptide analogue lirr, and/or at least one motif LIIRR (SEQ ID NO: 21) is replaced by its D-peptide analogue Him and/or at least one motif RRILL (SEQ ID NO: 33) is replaced by its D-peptide analogue rrill, and/or at least one motif RRLL (SEQ ID NO: 34) is replaced by its D-peptide analogue rrll,
and optionally the lysine moiety K is replaced by its D-amino acid analogue k or the lysine moiety (εK) is replaced by its D-amino acid analogue (εk),
a retro-inverso analogue thereof; and
a peptidomimetic analogue thereof.

In a preferred embodiment, the sequence motif A12 is selected from the group consisting of:

(SEQ ID NO: 6)
RRILKRRIL;
(SEQ ID NO: 7)
RRIL(εK)RRIL;
(SEQ ID NO: 8)
RRIIL(εK)RRIL;
(SEQ ID NO: 9)
RRILKRRIIL;
(SEQ ID NO: 10)
RRIILKRRIL;
(SEQ ID NO: 11)
RRIILKRRIIL;
(SEQ ID NO: 12)
RRIL(εK)RRIIL;
(SEQ ID NO: 13)
RRIIL(εK)RRIIL;
(SEQ ID NO: 16)
LIRRKRRIL;
(SEQ ID NO: 17)
LIRR(εK)RRIL;
(SEQ ID NO: 18)
RRILKLIIRR;
(SEQ ID NO: 28)
LIRRK(X⁴)RRIL,

wherein X⁴ is a sequence of four or five consecutive amino acid moieties conjugated to the epsilon amino group of the lysine moiety, preferably is a sequence of SEQ ID NO: 23 or SEQ ID NO: 24;
and a D-peptide analogue of any of these sequences of SEQ ID NO: 6-13, 16-18 or 28, wherein in said D-peptide analogue at least one motif RRIL (SEQ ID NO: 4) is replaced by its D-peptide analogue rril, and/or at least one motif RRIIL (SEQ ID NO: 5) is replaced by its D-peptide analogue rriil, and/or at least one motif LIRR (SEQ ID NO: 19) is replaced by its D-peptide analogue lirr, and/or at least one motif LIIRR (SEQ ID NO: 21) is replaced by its D-peptide analogue liirr, and optionally the lysine moiety K is replaced by its D-amino acid analogue k or the lysine moiety (εK) is replaced by its D-amino acid analogue (εk),
a retro-inverso analogue thereof; and
a peptidomimetic analogue thereof.

X⁴ may preferably selected from the group consisting of RRIL (SEQ ID NO: 4), RRIIL (SEQ ID NO: 5), LIRR (SEQ ID NO: 19), LIIRR (SEQ ID NO: 21), rril (D-analogue of SEQ ID NO: 4), rriil (D-analogue of SEQ ID NO: 5), lirr(D-analogue of SEQ ID NO: 19), liirr (D-analogue of SEQ ID NO: 21, and peptidomimetic analogues of any thereof. More preferably, X⁴ is RRIL (SEQ ID NO: 4) or LIRR (SEQ ID NO: 19). This means that either the C-terminal arginine moiety or the C-terminal leucine moiety may be conjugated to the epsilon amino group of the lysine moiety of X⁴.

The sequence motifs A1 usable in the sequence motifs A1 such as, e.g., those selected from the list consisting of

(SEQ ID NO: 4)
RRIL,
(SEQ ID NO: 5)
RRIIL,
(SEQ ID NO: 19)
LIRR,
(SEQ ID NO: 21)
LIIRR,
(SEQ ID NO: 33)
RRILL,
(SEQ ID NO: 34)
RRLL,
(D-analogue of SEQ ID NO: 4)
rril,
(D-analogue of SEQ ID NO: 5)
rriil,
(D-analogue of SEQ ID NO: 19)
lirr,
(D-analogue of SEQ ID NO: 21
liirr,
(D-analogue of SEQ ID NO: 33)
rrill,
(D-analogue of SEQ ID NO: 34)
rrll,

and
peptidomimetic analogues of any thereof
are freely combinable with another.

The sequence motifs A1 usable in the sequence motifs A1 such as, e.g., those selected from the list consisting of

### RRIL (SEQ ID NO: 4),

### RRIIL (SEQ ID NO: 5),

### LIRR (SEQ ID NO: 19),

### LIIRR (SEQ ID NO: 21),

rril (D-analogue of SEQ ID NO: 4),
rriil (D-analogue of SEQ ID NO: 5),
lirr (D-analogue of SEQ ID NO: 19),
liirr (D-analogue of SEQ ID NO: 21, and
peptidomimetic analogues of any thereof
are freely combinable with another.

Accordingly, in a more preferred embodiment, the sequence motif A1 is selected from the group consisting of (peptide No.):

### 1: RRILKRRIL (SEQ ID NO: 6);

2: rrilkrril (D-amino acid equivalent to SEQ ID NO: 6);

### 3: RRIL(εK)RRIL (SEQ ID NO: 7);

4: rril(εk)rril (D-amino acid equivalent to SEQ ID NO: 7);
5: rriIKRRIL (mixed LI D-amino acid equivalent to SEQ ID NO: 6);
6: rril(εK)RRIL (mixed LI D-amino acid equivalent to SEQ ID NO: 7);
7: rril(εK)RRIIL (mixed LI D-amino acid equivalent to SEQ ID NO: 12);
8: rriil(εK)RRIL (mixed LI D-amino acid equivalent to SEQ ID NO: 8);

### 9: RRIIL(εK)RRIL (SEQ ID NO: 8);

### 10: LIRRKRRIL (SEQ ID NO: 16);

### 11: LIRR(εK)RRIL (SEQ ID NO: 17);

### 12: LIRRK(X⁴)RRIL (SEQ ID NO: 28);

13: lirrKRRIL (mixed LI D-amino acid equivalent to SEQ ID NO: 16);
14: lirr(εK)RRIL (mixed LI D-amino acid equivalent to SEQ ID NO: 17);
15: lirrkrril (D-amino acid equivalent to SEQ ID NO: 16);
16: lirr(εk)rril (D-amino acid equivalent to SEQ ID NO: 17);
17: rriilkrriil (D-amino acid equivalent to SEQ ID NO: 11);
18: rril(εk)rriil (D-amino acid equivalent to SEQ ID NO: 12);
19: rriil(εk)rril (mixed LI D-amino acid equivalent to SEQ ID NO: 8);
20: rriil(εk)rriil (D-amino acid equivalent to SEQ ID NO: 13);
21: RRILkrriil (mixed LI D-amino acid equivalent to SEQ ID NO: 9);
22: RRILKrriil (mixed LI D-amino acid equivalent to SEQ ID NO: 9);
24: RRIILKrril (mixed LI D-amino acid equivalent to SEQ ID NO: 10);
25: RRIILkrriil (mixed LI D-amino acid equivalent to SEQ ID NO: 11);
26: RRIILKrriil (mixed LI D-amino acid equivalent to SEQ ID NO: 11);
27: RRIL(εk)rriil (mixed LI D-amino acid equivalent to SEQ ID NO: 12);
28: RRIL(εK)rriil (mixed LI D-amino acid equivalent to SEQ ID NO: 12);
29: RRIIL(εk)rril (mixed LI D-amino acid equivalent to SEQ ID NO: 8);
30: RRIIL(εK)rril (mixed LI D-amino acid equivalent to SEQ ID NO: 8);
31: RRIIL(εk)rriil (mixed LI D-amino acid equivalent to SEQ ID NO: 13);
32: RRIIL(εK)rriil (mixed LI D-amino acid equivalent to SEQ ID NO: 13);
33: rriIKRRIL (mixed LI D-amino acid equivalent to SEQ ID NO: 8);
34: rril(εk)RRIL (mixed LI D-amino acid equivalent to SEQ ID NO: 8);
35: rril(εk)RRIIL (mixed LI D-amino acid equivalent to SEQ ID NO: 8);
36: rriil(εk)RRIL (mixed LI D-amino acid equivalent to SEQ ID NO: 8);
37: rriil(εk)RRIIL (mixed LI D-amino acid equivalent to SEQ ID NO: 13);
38: rriil(εK)RRIIL (mixed LI D-amino acid equivalent to SEQ ID NO: 13);
39: rrilkRRIIL (mixed LI D-amino acid equivalent to SEQ ID NO: 9);
40: rriIKRRIIL (mixed LI D-amino acid equivalent to SEQ ID NO: 9);
41: rriilkRRIL (mixed LI D-amino acid equivalent to SEQ ID NO: 10);
42: rriilKRRIL (mixed LI D-amino acid equivalent to SEQ ID NO: 10);
43: rriilkRRIIL (mixed LI D-amino acid equivalent to SEQ ID NO: 11);
44: rriilKRRIIL (mixed LI D-amino acid equivalent to SEQ ID NO: 11);
45: rriIKRRIL (mixed LI D-amino acid equivalent to SEQ ID NO: 6);

### 46: RRILKRRIIL (SEQ ID NO: 9);

### 47: RRIILKRRIL (SEQ ID NO: 10);

### 48: RRIILKRRIIL (SEQ ID NO: 11);

### 49: RRIL(εK)RRIIL (SEQ ID NO: 12);

### 50: RRIIL(εK)RRIIL (SEQ ID NO: 13);

51: rrilkrriil (D-amino acid equivalent to SEQ ID NO: 9);
52: rriilkrril (D-amino acid equivalent to SEQ ID NO: 10);
53: lirrK(LIRR)RRIL (mixed LI D-amino acid equivalent to SEQ ID NO: 18);
54: lirrk(LIRR)RRIL (mixed LI D-amino acid equivalent to SEQ ID NO: 18);
55: LIRRK(lirr)RRIL (mixed LI D-amino acid equivalent to SEQ ID NO: 18);
56: LIRRK(LIRR)rril (mixed LI D-amino acid equivalent to SEQ ID NO: 18);

### 57: RRILKLIIRR (SEQ ID NO: 18);

58: rrilKLIIRR (mixed LI D-amino acid equivalent to SEQ ID NO: 18);
59: rrilkLIIRR (mixed LI D-amino acid equivalent to SEQ ID NO: 18);
60: RRILKIiirr (mixed LI D-amino acid equivalent to SEQ ID NO: 18);
61: rrilkliirr (D-amino acid equivalent to SEQ ID NO: 18);
62: RRILkliirr (mixed LI D-amino acid equivalent to SEQ ID NO: 18).

### 63: RRILL(εK)RRIL (SEQ ID NO: 29);

64: rrill(εK)RRIL (mixed LI D-amino acid equivalent to SEQ ID NO: 29);

### 65: RRIIL(εK)RRLL (SEQ ID NO: 30);

66: rriil(εK)RRLL (mixed LI D-amino acid equivalent to SEQ ID NO: 30); and

### 67: RRIL(PEG)₃RRIL (SEQ ID NO: 31); and

In a preferred embodiment, the sequence motif A1 is selected from the group consisting of peptide Nos. 1-62 as depicted above.

The peptide structure of peptides Nos. 1-9 may be depicted as follows, highlighting that the N-terminal amino group of the peptide moiety that forms the C-terminal peptide moiety forms an amide bond with the central lysyl moiety:

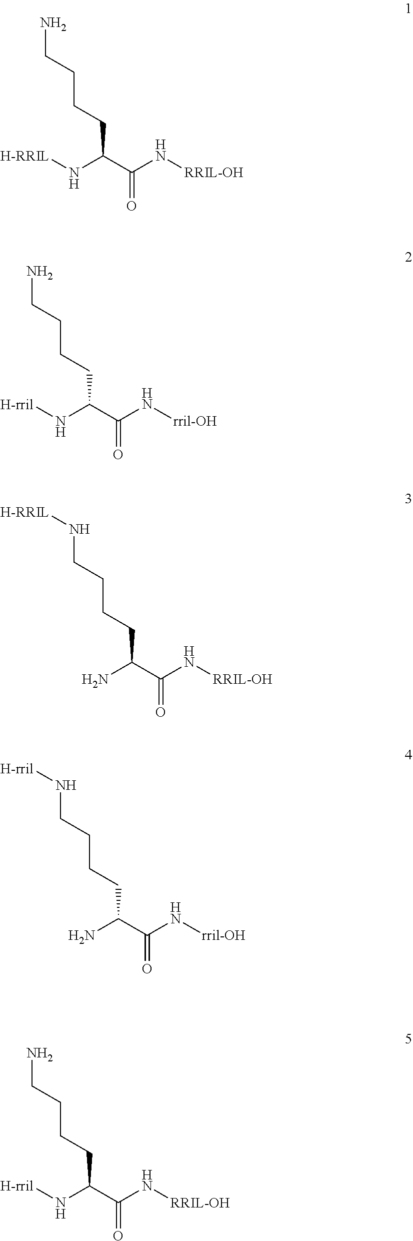

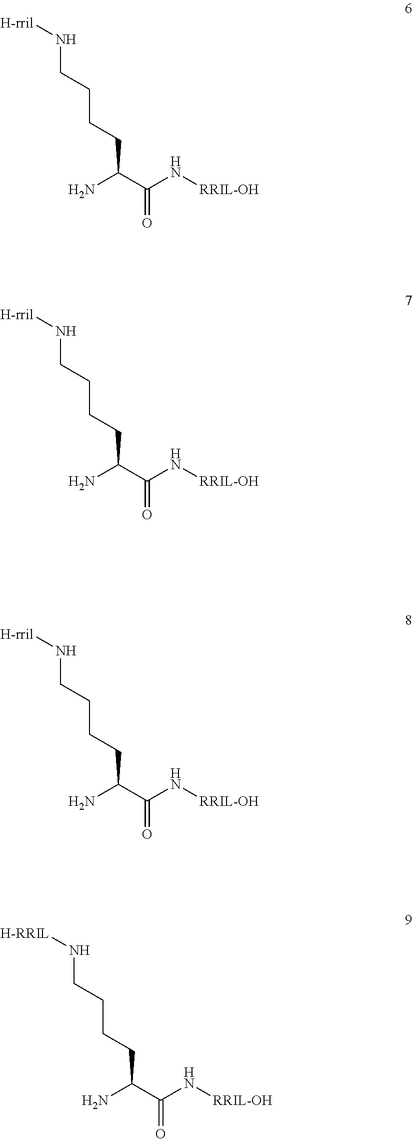

The corresponding structures without explicitly depicting the amino group are depicted in FIG. 1.

It will be understood that also any other combination may be used.

In an even more preferred embodiment, the peptide residue (A1) is selected from the group consisting of any of peptides 1-16, in particular selected from the group consisting of any of peptides 1-9 as indicated above.

The term “antigen of interest” may be understood in the broadest sense as any molecular structure that provokes a desired selective immune response directed towards said antigen. In principle, the antigen of interest B may be any molecular structure suitable for this purpose. Preferably, the antigen of interest B will be selected from the group consisting of (poly)peptides, (poly)saccharides, nucleotides, lipids, conjugates of two or more thereof and smaller molecular structures that may serve as a haptene.

Preferably, the antigen of interest is a defined antigen that induces a selective immune response directed to this antigen only. In this context, the term “of interest” indicates that it is not an arbitrary immune response that is achieved but rather a specifically chosen immune response. In particular, the peptidic entity A preferably has no immunogenic properties, i.e., does not serve as antigen. Preferably, the antigen of interest B is the predominant immunogenic molecular structure in the vaccine composition V, in other words, is the strongest antigen. Particularly preferably, the antigen of interest B is (essentially) the only immunogenic molecular structure in the vaccine composition V.

Therefore, the antigen will preferably not be large in size, typically bearing a molecular weight of not more than 5000 Da, more preferably of 250 to 2500 Da, in particular of 500 to 2000 Da. Such antigen of interest B which bears a molecular weight of not more than 5000 Da, more preferably of 250 to 2500 Da, in particular of 500 to 2000 Da, may also be designated as subunit vaccine.

In a preferred embodiment, the antigen of interest B is selected from the group consisting of a peptidic entity, a nucleic acid entity, an oligo- or polysaccharide entity, and a combination or conjugate of two or more thereof.

Highly preferably, the antigen of interest B is a peptidic entity. Particularly preferably, the antigen of interest B is a short peptidic entity of (approximately) 5 to 20 amino acid moieties, in particular 8 to 12 amino acid moieties. Such antigen of interest B which is a short peptidic entity of (approximately) 5 to 20 amino acid moieties, in particular 8 to 12 amino acid moieties, may also be designated as subunit vaccine.

The antigen of interest B may be an independent molecular structure or may be conjugated with the peptidic entity A. This may be a covalent or non-covalent linkage, preferably a covalent linkage. Optionally, the covalent or non-covalent linkage may be cleavable under certain conditions. Exemplarily, it may be optionally cleavable under reductive environments (e.g., when the linkage is a disulfide bond) or may be cleavable by enzymatic cleavage (when it is a cleavage site of an enzyme).

In a preferred embodiment, the peptidic entity A and the antigen of interest B are conjugated with another.

In a particularly preferred embodiment, the peptidic entity A and the antigen of interest B are both peptidic entities conjugated with another.

This may be achieved by any means in the art. If the peptidic entity A as well as the antigen of interest B are both consisting of natural L-amino acid moieties (optionally subjected to one or more post-translational modifications), a peptide strand comprising both the peptidic entity A and the antigen of interest B may be expressed by an organism. The peptidic entity A may then be N-terminally or C-terminally of the antigen of interest B. The peptide strand may optionally comprise one or more further amino acid moieties.

Likewise, if the peptidic entity A as well as the antigen of interest B are both consisting of amino acid moieties and analogues thereof that can be used in peptide synthesis (e.g., SPPS or LPPS), the a peptide strand comprising both the peptidic entity A and the antigen of interest B may be synthesized. The peptidic entity A may then be N-terminally or C-terminally of the antigen of interest B. The peptide strand may optionally comprise one or more further amino acid moieties or subsequent modifications (e.g., acylation, acetylation etc.).

Independ on whether also the antigen of interest B is also a peptidic structure, orthogonal conjugation may be used. This may be based on the selective removal of protecting groups. Alternatively or additionally, selective conjugation techniques such as Native Chemical Ligation (NCL), Click Chemistry, Maleimide-Thiol Conjugation, enzymatic conjugation, biochemical protein ligation (e.g., based on enzymes) and/or soluble handling conjugation may be used.

If a selective conjugation method is use, additional moieties may be added to the sequence motif A1. If, for example NCL is used as for orthogonal conjugation, an N-terminal cysteine moiety may be added to the sequence motif A1. Then, the molecular structure to be conjugated may be bear a thiol moiety. Alternatively, at the C-terminus of the sequence motif A1, a thiol moiety may be present. Then, the molecular structure to be conjugated may be bear an N-terminal cysteine moiety 8 also designatable as cysteinyl moiety, cysteine residue, cysteinyl residue, Cys).

The person skilled in the art will be aware of the details usable in each conjugation method.

In a more preferred embodiment, the peptidic entity A and the antigen of interest B together form a peptide strand AB.

Preferably the peptidic entity A and the antigen of interest B together form a peptide strand AB of not more than 50 amino acid moieties, preferably not more than 40 amino acid moieties, in particular not more than 30 amino acid moieties in length.

In principle, the silica particles C may be obtained by any means. As the peptidic entity A is selectively precipitating silica from a solution comprising silicic acid, the particles will preferably be obtained by such means.

Accordingly, in a preferred embodiment, the silica particles C have been obtained by precipitating silica from a solution comprising silicic acid in the presence of the peptidic entity A and preferably the antigen of interest B.

Such precipitated silica particles C bear special structural characteristics which differ from silica particles obtained by other means such as, e.g., milling or precipitating by mere chemical means. Exemplarily, the silica particles C are particularly porous which improves their usability as adjuvants. Further, the form of the silica particles C can be very well adjusted by means of choosing a specific peptide sequence. As experimentally demonstrated, well symmetric sprees and rod-like structures may be obtained. The particle size may be adjusted very well as well.

In a preferred embodiment, the silica particles C bear spherical or rod-like structures having mean diameters of 0.1 to 10 μm, in particular 0.5 to 2 μm (determined by SEM). Alternatively, also sheet-like structures may be obtainable.

Preferably, the silica particles C described herein are mesoporous silica particles, i.e., contains pores in the mean diameter range of from 2 to 50 nm by microscopy (e.g., scanning electron microscopy, SEM).

As used herein, an mean diameter is referred to is typically the weighted arithmetic mean as preferably determined by microscopy (e.g., scanning electron microscopy, SEM). It will be understood that a particle s per definition a solid component that is at least 10 nm in size. Accordingly, the average as used herein is preferably the weighted arithmetic mean of all particles of 10 nm or more in size.

The loading of the silica particles C with components A+B may be adapted to the purpose intended. In a preferred embodiment, the mass ratio of (components A+B):silica in the silica particles C is in the range of from 0.1:99.9 to 60:40, preferably in the range of from 1:99 to 55:45, in particular in the range of from 5:95 to 50:50.

As indicated above, the special characteristics of the silica particles C embracing the peptidic entity A and the antigen of interest B which are usable as or in a vaccine composition V is the use of the peptidic entity A for preparing the silica particles C embracing the peptidic entity A and the antigen of interest B.

Accordingly, a further aspect of the present invention relates to a peptidic entity A comprising (or consisting of):
(A1) at least one first sequence motif A1 of a sequence

(SEQ ID NO: 23)
RR-X^{1a}-X^{1b}-L,

wherein
X^{1a} is a hydrophobic amino acid moiety,
X^{1b} is a peptide bond or a hydrophobic amino acid moiety,
or a D-peptide analogue of the sequence SEQ ID NO: 23, wherein one or more of the L-amino acid moieties is/are replaced by the respective D-amino acid(s),
or a retro-inverso analogue of the sequence of SEQ ID NO: 23,
or a peptidomimetic analogue of the sequence of SEQ ID NO: 23; and
(A2) at least one second sequence motif A2 of a sequence selected from the group consisting of

(SEQ ID NO: 23)
RR-X^{1a}-X^{1b}-L,
and
(SEQ ID NO: 24)
L-X^{1c}-X^{1d}-RR,

wherein
X^{1a} is a hydrophobic amino acid moiety,
X^{1b} is a peptide bond or a hydrophobic amino acid moiety,
X^{1c} is a hydrophobic amino acid moiety,
X^{1d} is a bond or a hydrophobic amino acid moiety,
or a D-peptide analogue of the sequence SEQ ID NO: 23 or 24, wherein one or more of the L-amino acid moieties is/are replaced by the respective D-amino acid(s),
or a retro-inverso analogue of the sequence of SEQ ID NO: 23 or 24,
or a peptidomimetic analogue of the sequence of SEQ ID NO: 23 or 24.

It will be understood that all definitions are preferred embodiments as indicated above mutatis mutandis also applies to the peptidic entity as such.

In a more preferred embodiment, the peptidic entity A is defined as above in the context of the vaccine composition V.

More generally, the present invention also relates to a peptidic entity A comprising at least two sequence motifs each selected independently from another from the group consisting of

(SEQ ID NO: 23)
RR-X^{1a}-X^{1b}-L,
and
(SEQ ID NO: 24)
L-X^{1c}-X^{1d}-RR,

wherein
X^{1a} is a hydrophobic amino acid moiety,
X^{1b} is a peptide bond or a hydrophobic amino acid moiety,
X^{1c} is a hydrophobic amino acid moiety,
X^{1d} is a bond or a hydrophobic amino acid moiety,
or a D-peptide analogue of the sequence SEQ ID NO: 23 or 24, wherein one or more of the L-amino acid moieties is/are replaced by the respective D-amino acid(s),
or a retro-inverso analogue of the sequence of SEQ ID NO: 23 or 24,
or a peptidomimetic analogue of the sequence of SEQ ID NO: 23 or 24.

In a particularly preferred embodiment, the peptidic entity A comprising a sequence motif A12 having the sequence

(SEQ ID NO: 15)
RRI-X¹-L-X²-RRI-X³-L
or
(SEQ ID NO: 20)
L-X¹-IRR-X²-RRI-X³-L,

wherein each X¹ and X³ are independently from another either a peptide bond or an isoleucine moiety, and X² is a peptide moiety optionally substituted by a further sequence motif A3 which is equal to sequence motif A2, preferably wherein said X2 is a peptide moiety is one amino acid moiety optionally substituted by a further sequence motif A3 which is equal to sequence motif A2,
or a D-peptide analogue thereof, wherein one or more of the L-amino acid moieties is/are replaced by the respective D-amino acid(s).

Accordingly, in a preferred embodiment, the sequence motif A12 is selected from the group consisting of:

(SEQ ID NO: 6)
RRILKRRIL;
(SEQ ID NO: 7)
RRIL(εK)RRIL;
(SEQ ID NO: 8)
RRIIL(εK)RRIL;
(SEQ ID NO: 9)
RRILKRRIIL;
(SEQ ID NO: 10)
RRIILKRRIL;
(SEQ ID NO: 11)
RRIILKRRIIL;
(SEQ ID NO: 12)
RRIL(εK)RRIIL;
(SEQ ID NO: 13)
RRIIL(εK)RRIIL;
(SEQ ID NO: 16)
LIRRKRRIL;
(SEQ ID NO: 17)
LIRR(εK)RRIL;
(SEQ ID NO: 18)
RRILKLIIRR;
(SEQ ID NO: 29)
RRILL(εK)RRIL;
(SEQ ID NO: 30)
RRIIL(εK)RRLL;
(SEQ ID NO: 31)
RRIL(PEG)₃RRIL;
(SEQ ID NO: 28)
LIRRK(X⁴)RRIL,

wherein X⁴ is a sequence of four or five consecutive amino acid moieties conjugated to the epsilon amino group of the lysine moiety, preferably is a sequence of SEQ ID NO: 23 or SEQ ID NO: 24; and a D-peptide analogue of any of these sequences of SEQ ID NO: 6-13, 16-18 or 28, wherein in said D-peptide analogue at least one motif RRIL (SEQ ID NO: 4) is replaced by its D-peptide analogue rril, and/or at least one motif RRIIL (SEQ ID NO: 5) is replaced by its D-peptide analogue rriil, and/or at least one motif LIRR (SEQ ID NO: 19) is replaced by its D-peptide analogue lirr, and/or at least one motif LIIRR (SEQ ID NO: 21) is replaced by its D-peptide analogue Him and/or at least one motif RRILL (SEQ ID NO: 33) is replaced by its D-peptide analogue rrill, and/or at least one motif RRLL (SEQ ID NO: 34) is replaced by its D-peptide analogue rrll,
and optionally the lysine moiety K is replaced by its D-amino acid analogue k or the lysine moiety (εK) is replaced by its D-amino acid analogue (εk),
a retro-inverso analogue thereof; and
a peptidomimetic analogue thereof.

Further preferred embodiments are laid out above.

As indicated above, these peptidic entity may be very well be used for preparing silica particles C embracing the peptidic entity A and the antigen of interest B which are usable as or in a vaccine composition V as described herein. The peptidic entity may however also be used for any other purpose. In particular, the peptidic entity according to the invention may be used to prepare silica particles C in general, which optionally comprise an antigen of interest B but do not necessarily comprise an antigen of interest B.

Accordingly, a further aspect of the present invention relates to a silica particle C comprising silica and a peptidic entity comprising a sequence motif A12 according to the present invention, preferably wherein said silica particle C:

is obtained by precipitating silica from a solution comprising silicic acid in the presence of the peptidic entity A, and/or
bears spherical or rod-like structures having mean diameters of 0.1 to 10 μm, in particular 0.5 to 2 μm;
in particular wherein said silica particle C is a silica particle C according to the present invention.

A still further aspect of the present invention relates to a silica particle C comprising silica and a peptidic entity of not more than 50 amino acid moieties comprising at least one sequence motif A1 of a sequence

(SEQ ID NO: 23)
RR-X^{1a}-X^{1b}-L,

wherein
X^{1a} is a hydrophobic amino acid moiety,
X^{1b} is a peptide bond or a hydrophobic amino acid moiety,
or a D-peptide analogue of the sequence SEQ ID NO: 23, wherein one or more of the L-amino acid moieties is/are replaced by the respective D-amino acid(s),
or a retro-inverso analogue of the sequence of SEQ ID NO: 23,
or a peptidomimetic analogue of the sequence of SEQ ID NO: 23,
preferably wherein said silica particle C:
is obtained by precipitating silica from a solution comprising silicic acid in the presence of the peptidic entity A, and/or
bears spherical or rod-like structures having mean diameters of 0.1 to 10 μm, in particular 0.5 to 2 μm,
in particular wherein said silica particle C is a silica particle C according to the present invention.

It will be understood that all definitions are preferred embodiments as indicated above mutatis mutandis also applies to the silica particle C.

Such silica particle C may optionally comprise any cargo which may be selected from the group consisting of small-molecule compounds (e.g., pharmaceutically active agents, dyes, diagnostic probes etc.), short peptides, proteins, polysaccharides etc.

The silica particle C may be used as a drug carrier. Then, other compounds may be loaded as a cargo.

As indicated above, as far as the silica particle C embraces an antigen of interest, it may be used for vaccination. Then, the silica particle C is a silica particle C which may as such serve as an adjuvant in vaccination. The silica particle C (typically inherently embracing the peptidic entity A) and further the embracing the antigen of interest B may be used as a vaccine composition, i.e., as a vaccine.

Accordingly, a sill further aspect of the present invention relates to the use of a vaccine composition V according to the present invention for vaccination.

In other words, the invention relates to a vaccine composition V according to the present invention for use in a method for vaccination or a patient in need thereof.

In other words, the invention relates to a method of vaccination in a patient in need thereof, said method comprising administering a sufficient amount of a vaccine composition V according to the present invention to said patient.

A sill further aspect of the present invention relates to the use of a silica particle C according to the present invention as an adjuvant in vaccination.

In more general terms, the present invention relates to the use of a silica particle C embracing a silaffin-derived peptide as an adjuvant in vaccination.

It will be understood that the definitions and preferred embodiments as laid out above optionally also apply to such use. Preferably, the silica particles C are such as described above.

Further, the present invention relates to the use of a silica particle C embracing a silaffin-derived peptide and an antigen of interest B (i.e., preferably a subunit vaccine) as vaccine.

In other words, the invention relates to a silica particle C embracing a silaffin-derived peptide and an antigen of interest B (i.e., preferably a subunit vaccine) for use in a method for vaccination or a patient in need thereof.

In other words, the invention relates to a method of vaccination in a patient in need thereof, said method comprising administering a sufficient amount of a silica particle C embracing a silaffin-derived peptide and an antigen of interest B (i.e., preferably a subunit vaccine) to said patient.

It will be understood that the definitions and preferred embodiments as laid out above optionally also apply to such use.

As used herein, “silaffin-derived peptide” (usable for use for vaccination) may be understood in the broadest sense as any peptidic entity of at least four consecutive amino acid moieties that is homologue to a silaffin protein or has one amino acid moiety inserted into a consecutive sequence of at least four amino acid moieties that is homologue to a silaffin protein. Typically, in the sense of the present invention, the silaffin-derived peptide facilitates the generation of silica particles.

Preferably, silaffin is silaffin-1 (from Cylindrotheca fusiformis) having at least 90% sequence homology, more preferably at least 95% sequence homology, even more preferably at least 99% sequence homology, in particular sequence identity to

SEQ ID NO: 22:
MKLTAIFPLLFTAVGYCAAQSIADLAAANLSTEDSKSAQLISADSSDDAS
DSSVESVDAASSDVSGSSVESVDVSGSSLESVDVSGSSLESVDDSSEDSE
EEELRILSSKKSGSYYSYGTKKSGSYSGYSTKKSASRRILSSKKSGSYSG
YSTKKSGSRRILSSKKSGSYSGSKGSKRRILSSKKSGSYSGSKGSKRRNL
SSKKSGSYSGSKGSKRRILSSKKSGSYSGSKGSKRRNLSSKKSGSYSGSK
GSKRRILSGGLRGSM.

More preferably, the silaffin-derived peptide (usable for use for vaccination) is a truncated peptide of between four and ten amino acid moieties length, in particular four or five amino acid moieties length, of the sequence of SEQ ID NO: 22 or a sequence that has at least 95% homology to the sequence of SEQ ID NO: 22,

or a D-peptide analogue thereof, wherein one or more of the L-amino acid moieties is/are replaced by the respective D-amino acid(s),
or a retro-inverso analogue of the sequence of SEQ ID NO: 22 or a sequence that has at least 95% homology to the sequence of SEQ ID NO: 22,
or a peptidomimetic analogue of the sequence of SEQ ID NO: 22 or a sequence that has at least 95% homology to the sequence of SEQ ID NO: 22.

Even more preferably, the silaffin-derived peptide (usable for use for vaccination) is a truncated peptide of between four and ten amino acid moieties length, in particular four or five amino acid moieties length of a peptide that has at least 90% sequence homology, more preferably at least 95% sequence homology, even more preferably at least 99% sequence homology, in particular sequence identity to the silaffin peptide R5 (sequence: SSKKSGSYSGSKGSKRRIL (SEQ ID NO: 1))

or a D-peptide analogue thereof, wherein one or more of the L-amino acid moieties is/are replaced by the respective D-amino acid(s),
or a retro-inverso analogue of the sequence of SEQ ID NO: 1 or a sequence that has at least 95% homology to the sequence of SEQ ID NO: 1,
or a peptidomimetic analogue of the sequence of SEQ ID NO: 1 or a sequence that has at least 95% homology to the sequence of SEQ ID NO: 1.

In a highly preferred embodiment, the silaffin-derived peptide (usable for use for vaccination) is a peptide, preferably comprising up to 30 amino acids, preferably up to 25 amino acids, in particular up to 20 amino acids, comprising one, two or more peptide moieties independently from another selected from the list consisting of:

(SEQ ID NO: 23)
RR-X^{1a}-X^{1b}-L;
and
(SEQ ID NO: 24)
L-X^{1c}-X^{1d}-RR,

wherein
X^{1a} is a hydrophobic amino acid moiety, preferably wherein said hydrophobic amino acid moiety is selected from the group consisting of Ile, Leu and Val, more preferably is Ile or Leu, in particular is Ile,
X^{1b} is a peptide bond or a hydrophobic amino acid moiety, preferably wherein said hydrophobic amino acid moiety is selected from the group consisting of Ile, Leu and Val, more preferably is Ile or Leu, in particular is Ile,
X^{1c} is a hydrophobic amino acid moiety, preferably wherein said hydrophobic amino acid moiety is selected from the group consisting of Ile, Leu and Val, more preferably is Ile or Leu, in particular is Ile,
X^{1d} is a bond or a hydrophobic amino acid moiety, preferably wherein said hydrophobic amino acid moiety is selected from the group consisting of Ile, Leu and Val, more preferably is Ile or Leu, in particular is Ile;
a D-peptide analogue of the sequence SEQ ID NO: 23 or 24, wherein one or more of the L-amino acid moieties is/are replaced by the respective D-amino acid(s);
a retro-inverso analogue of the sequence of SEQ ID NO: 23 or 24; and
a peptidomimetic analogue of the sequence of SEQ ID NO: 23 or 24;

Accordingly, a silaffin-derived peptide (usable for use for vaccination) can also be such comprising a single peptide motif A1 and/or A2 as described above. As mentioned before all definitions and preferred embodiments as laid out above optionally also apply to such use

The present invention also relates to the use of a peptidic entity A for preparing a silica particle C.

Highly preferably, the silaffin-derived peptide (usable for use for vaccination) is a peptidic entity A as described above, in particular such according to RRI-X¹-L (SEQ ID NO: 3), wherein X¹ is either a peptide bond or an isoleucine moiety,

or a D-peptide analogue thereof, wherein one or more of the L-amino acid moieties is/are replaced by the respective D-amino acid(s),
or a retro-inverso analogue of the sequence of SEQ ID NO: 3,
or a peptidomimetic analogue of the sequence of SEQ ID NO: 3.

Alternatively, silaffin-derived peptide (usable for use for vaccination) may be the expression product of Gene ID: 7445519 (from Thalassiosira pseudonana) or any other silaffin protein, in particular a truncated peptide thereof of between four and ten amino acid moieties length, in particular four or five amino acid moieties length or a D-peptide, retro-inverso or a peptidomimetic analogue thereof.

As described herein, the peptidic entity A of the present invention may be used to particularly preferably prepare silica particles C, in a particularly beneficial and stream-lined method.

Accordingly, a sill further aspect of the present invention relates to a method for preparing a vaccine composition V according to the present invention, comprising the following steps:
(i) providing the following:
   (a) a peptidic entity A according to the present invention,
   (b) an antigen of interest B according to the present invention, wherein said peptidic entity A and said antigen of interest B may optionally be conjugated with another, and
   (c) an aqueous solution SC comprising silicic acid;
(ii) dissolving the peptidic entity A and the antigen of interest B in the aqueous solution SC, thereby forming solution S;
(iii) incubating the solution S obtained from step (ii) under conditions allowing the precipitation of silica particles C embracing the peptidic entity A and the antigen of interest B;
(iv) optionally separating the silica particles C obtained from step (iii) from the solution S;
(v) optionally washing the silica particles C obtained from any of steps (iii) or (iv),
(vi) optionally drying the silica particles C obtained from any of steps (iii) to (v); and
(vii) optionally adding a pharmaceutically acceptable carrier to the silica particles C obtained from any of steps (iii) to (vi).

As indicated above, the silica particles can be very well but do not necessarily have to be leaded with an antigen of interest B.

Accordingly, a sill further aspect of the present invention relates to a method for preparing silica particle C according to the present invention, comprising the following steps:
(i) providing the following:
   (a) peptidic entity A comprising a sequence motif A12 according to the present invention,
   (b) optionally an antigen of interest B according to the present invention, wherein said peptidic entity A and said antigen of interest B, if present, may optionally be conjugated with another, and
   (c) an aqueous solution SC comprising silicic acid;
(ii) dissolving the peptidic entity A comprising a sequence motif A12 and, if present, the antigen of interest B in the aqueous solution SC, thereby forming solution S;
(iii) incubating the solution S obtained from step (ii) under conditions allowing the precipitation of silica particles C embracing the peptidic entity A and, if present, the antigen of interest B;
(iv) optionally separating the silica particles C obtained from step (iii) from the solution S;
(v) optionally washing the silica particles C obtained from any of steps (iii) or (iv);
(vi) optionally drying the silica particles C obtained from any of steps (iii) to (v); and
(vii) optionally adding a pharmaceutically acceptable carrier to the silica particles C obtained from any of steps (iii) to (vi).

It will be understood that all definitions are preferred embodiments as indicated above mutatis mutandis also applies to the methods.

As laid out above, providing the peptidic entity (A) (step (i)) may be performed by any means known in the art, preferably by SPPS, LPPS or a combination thereof, in particular by SPPS.

As laid out above, providing the antigen of interest B (step (i)) may be performed by any means known in the art and depending on the molecular structure of the antigen of interest B. If the antigen of interest is a peptidic entity, it may preferably be obtained by means of SPPS, LPPS or a combination thereof, in particular by SPPS. Alternatively, a peptidic antigen of interest B may be obtained by biotechnological means as described above.

As indicated above, in particular when the peptidic entity (A) and the antigen of interest B are both peptidic entities, these may be provided on a common peptide strand comprising A and B. A may be N-terminal of B or may be C-terminal of B.

Such common peptide strand AB comprising A and B may preferably obtained by synthesis of a common consecutive peptide strand AB, more preferably by means of a single consecutive synthesis by means of SPPS, LPPS or a combination thereof, in particular by means of SPPS.

Alternatively, a common peptide strand AB comprising A and B may be obtained by biotechnological means as described above, i.e., by (heterologous) expression of a fusion peptide AB comprising A and B in any orientation.

Alternatively, the peptidic entities A and B may be synthesized independently from another and subsequently conjugated, e.g., by means of e.g., Native Chemical Ligation (NCL), Click Chemistry, Maleimide-Thiol Conjugation, enzymatic conjugation, biochemical protein ligation and/or soluble handling conjugation. Alternatively, any other orthogonal conjugation method may be used.

A common conjugate comprising A and B may also be used for auto-encapsulation, i.e., the encapsulation of the conjugate in the later formed particles by itself during silica formation.

As laid out above, A and B do not necessarily have to be conjugated with another. Thus, A and B may also remain independent (unconjugated) molecular structures dissolvable in a common solution S.

Providing of an aqueous solution SC comprising silicic acid (step (i) may be obtained by any means. Preferably, due to the comparably low stability of silicic acid, silicic acid is generated just prior to the method of the present invention. Exemplarily, this may be achieved by hydrolysis of a precursor such as, e.g., tetramethoxysilane. As laid out in the Examples below, tetramethoxysilane may be hydrolyzed in an acidic environment (e.g., 0.5 to 1.5 mM HCl).

The components A and B are dissolved in the aqueous solution SC comprising silicic acid thereby forming the solution S comprising A, B and C.

The concentrations of the components A, B and C in the solution S may be adapted accordingly. Exemplarily, the solution S may comprise 0.1 to 1 mg/ml of peptidic entity A. Exemplarily, the solution S may comprise approximately 0.01 to 10 mg/ml of antigen of interest B. Exemplarily, the solution S may comprise 10 to 100 mM (i.e. approximately 1 to 10 mg/ml) of silicic acid.

Dissolving of the components A and B in the solution SC (step (ii) may be performed by any means, e.g., under stirring.

Incubating the solution S obtained from step (ii) under conditions allowing the precipitation of silica particles C may be performed under any conditions usable for this purpose. Exemplarily, the solution may be incubated at room temperature (RT) for a period of from 1 min to 24 h, preferably 5 min to 12 h, more preferably 10 min to 8 h, even more preferably 12 min to 4 h, even more preferably 15 min to 120 min, in particular 20 min to 60 min (e.g., approximately 30 min).

Then, the silica particles C are already obtainable. Optionally, the silica particles C obtained from step (iii) may be separated from the solution S (step (iv)). This may be performed by any means such as, e.g., centrifugation, filtration, cross-flow filtration, or a combination of two or more thereof.

Optionally, silica particles C obtained from any of steps (iii) or (iv) may be washed (step (v)). This may be performed by any means. Exemplarily, the silica particles C may be washed with buffer or with water.

Optionally, the silica particles C obtained from any of steps (iii) to (v) may be dried. This may be performed by any means. Exemplarily, the silica particles C may be dried in an exicator or in a warm gas/air stream. Drying may also include freeze-drying (lyophilization).

As indicated above, the compositions comprising the silica particle C (e.g., the vaccine composition V) may be pharmaceutical compositions. Accordingly, a pharmaceutically acceptable carrier may optionally be added to the silica particles C obtained from any of steps (iii) to (vi).

Accordingly, a sill further aspect of the present invention relates to the silica particles C according to the present invention for use as a medicament.

Accordingly, a sill further aspect of the present invention relates to the vaccine composition V according to the present invention for use as a medicament.

The silica particles C according to the present invention, in particular when forming a vaccine composition V and potentially forming part of a pharmaceutical composition may be used for various pharmaceutical purposes. In particular for vaccination purposes.

Accordingly, a sill further aspect of the present invention relates to the vaccine composition V according to the present invention for use in a method for preventing or treating a patient being of risk of or suffering from a pathologic condition associated with a pathogen or mutated cells bearing the antigen of interest B.

In other words, the present invention also relates to a method for preventing or treating a patient being of risk of or suffering from a pathologic condition associated with a pathogen or mutated cells bearing the antigen of interest B, said method comprising administration of the vaccine composition V according to the present invention in a sufficient concentration to said patient.

It will be understood that all definitions are preferred embodiments as indicated above mutatis mutandis also applies to the pharmaceutical use.

Accordingly, the present invention relates to the vaccine composition V according to the present invention for use in a method for preventing a patient being of risk of or suffering from a pathologic condition associated with a pathogen bearing the antigen of interest B.

Accordingly, the present invention relates to the vaccine composition V according to the present invention for use in a method for preventing a patient being of risk of or suffering from a pathologic condition associated with mutated cells bearing the antigen of interest B.

Accordingly, the present invention relates to the vaccine composition V according to the present invention for use in a method for treating a patient suffering from a pathologic condition associated with a pathogen bearing the antigen of interest B.

Accordingly, the present invention relates to the vaccine composition V according to the present invention for use in a method for treating a patient suffering from a pathologic condition associated with mutated cells bearing the antigen of interest B.

As used in the context of the present invention, the term “patient” may be understood in the broadest sense as any living being, which is preferably any animal, more preferably a mammal including human, in particular a human being.

The term “suffering from” as used herein may be understood in the broadest sense in a way that the patient has developed a pathological condition associated with pathogen or mutated cells bearing the antigen of interest B, i.e., that the antigen of interest B is present in the patient. The patient suffering from a disorder not necessarily but optionally bears medicinal symptoms.

The term “being at risk of” or “being at risk of developing” means that the patient has a certain risk of having a disorder associated a pathogen or mutated cells bearing the antigen of interest B.

Preferably, administration is systemic administration (e.g., intravenously (i.v.), intraarterially (i.a.), intraperitoneally (i.p.), intramusculary (i.m.), subcutaneously (s.c.), transdermally, nasally). Alternatively, administration may also be local administration (e.g., intrathecally or intravitreally). Preferably, administration is systemic administration, in particular intravenous injection.

Such pathologic condition may exemplarily be an infective disease such as Human Immunodeficiency Virus (HIV) infections, polio, human papilloma virus, malaria, etc. or may be a neoplasia such as cancer (e.g., metastatic cancer).

A pathogen may be any entity harming or being at risk of harming the patient's body. Exemplarily a pathogen may be selected from bacteria, viruses, fungi, protozoa, plasmodia, trypanosomae etc. Exemplarily, a pathogen may be Human Immunodeficiency Virus (HIV) polio virus, human papilloma virus, or plasmodium.

A mutated cell may exemplarily be a neoplastic cell, in particular a cancer cell, optionally a metastatic cancer cell.

It will be understood that vaccination may also be used for non-pharmaceutic purposes such as for the preparation of antibodies and/or research purposes.

A sill further aspect of the present invention relates to a method for producing an antibody of interest directed towards an antigen of interest B, said method comprising the steps:
(I) providing a non-human animal;
(II) applying a vaccine composition V comprising the antigen of interest B according to the present invention to the non-human animal;
(III) keeping the non-human animal vaccinated according to step (ii) under conditions allowing a secondary immune response directed towards the antigen of interest B comprised in the vaccine composition V; and
(IV) obtaining the antibody of interest from body fluids, in particular blood, of the non-human animal of step (iii).

It will be understood that all definitions are preferred embodiments as indicated above mutatis mutandis also applies to this method.

The routes of administration usable in step (II) have already been laid out in the context of the pharmaceutical use above.

The person skilled in the art will be aware of how to keep an animal to allow a secondary immune response directed towards the antigen of interest B. Typically, the antigen is injected once, twice, three times or more often and the animal is the kept for 1 to 6 weeks until the antibodies are obtained. This may or may not include scarifying the animal.

It will be understood that the antibody-producing cells may also be cultivated further in vitro and, optionally, may be fused with other cell types. This may allow obtaining monoclonal and/or humanized antibodies. Those skilled in the art will be aware of the respective methods.

In principle, the silica particles C may also be used for any other purposes such as, e.g., as drug carriers, as chromatographic stationary phase, etc. One of the benefits of the silica particles C of the present invention is their homogeneity in size and shape, their mesoporous characteristics and the absence of toxic agents.

In particular if the intended use of a silica particle C of the present invention is not the use as a for vaccination, the peptidic components, optionally also all organic components, may be removed from the obtained silica particles. This may be performed by means of heating the silica particles above 200° c. or above 500° C. or even above 1000° in order to remove the organic components. Then merkly inorganic mesoporous silica particles C* are obtainable.

The invention is further explained by the following Examples and Figures, which are intended to illustrate, but not to limit the scope of the present invention.

### BRIEF DESCRIPTION OF THE FIGURES

FIG. 1 shows the molecular structures of the divalent RRIL or RRIIL peptides comprising SEQ ID NO: 4 and/or 5. Herein, the capital letters indicate L-amino acid moieties, while the small letters (minuscule) indicate the respective D-amino acid moieties.

FIG. 2 shows SEM micrographs of the silica particles mineralised by peptides (A) 1 (according to SEQ ID NO: 6), (B) 2, (C) 3 (according to SEQ ID NO: 7), (D) 4, (E) 5, (F) 6, (G) 7, (H) 8, and (I) 9.

FIG. 3 shows TEM micrographs and CD spectra. FIG. 3A-D: TEM of the silica particles formed in the presence of peptides (A) 1 (according to SEQ ID NO: 6), (B, C) 8 and of peptide 8 in the absence of silicic acid/silica (D). FIG. 3E-G: CD of peptides (E) 1 and 2, (F) 3 (according to SEQ ID NO: 7) and 4, (G) 5, 6, 7, 8 and 9.

FIG. 4 shows transmission electron micrographs of peptides 1 (A) and 8 (B).

FIG. 5 shows silica particles the microscopic morphology of silica material precipitated with the silaffin peptide 64 (EHT: 5.00 kW, Mag: 20.00 KX, WD: 7.2 mm, Signal A: SE2). The scale bar indicates 200 nm.

FIG. 6 shows silica particles the microscopic morphology of silica material precipitated with the silaffin peptide 66 (EHT: 5.00 kW, Mag: 5.00 KX, WD: 7.1 mm, Signal A: SE2). The scale bar indicates 2 μm.

FIG. 7 shows silica particles the microscopic morphology of silica material precipitated with the silaffin peptide 67 (EHT: 5.00 kW, Mag: 5.00 KX, WD: 7.4 mm, Signal A: SE2). The scale bar indicates 1 μm.

FIG. 8 shows silica particles the microscopic morphology of silica material precipitated with the silaffin peptide 68 (EHT: 5.00 kW, Mag: 5.00 KX, WD: 7.2 mm, Signal A: InLens). The scale bar indicates 1 μm.

FIG. 9 shows the molecular structures of the divalent peptides 64, 66 and 67. Herein, the capital letters indicate L-amino acid moieties, while the small letters (minuscule) indicate the respective D-amino acid moieties. In the structure of peptide 67, the N-terminal amino moiety of the motif RRIL (SEQ ID NO: 4) is depicted to show that the binding site is the N-terminal amino moiety of this motif.

### EXAMPLES

### Example 1—Testing a Library of Peptides Comprising a RRIL (SEQ ID NO: 4) and RRIIL (SEQ ID NO: 5) Motif for Preparing Silica Particles

In this Example, we describe the synthesis and analysis of a library of peptides with RRIL (SEQ ID NO: 4) and RRIIL (SEQ ID NO: 5) motifs consisting of L- and D-amino acid moieties and assess their silica precipitating properties. A library of linear and branched peptides with two RRIL (SEQ ID NO: 4) motifs, derived from R5 silaffins, were explored for their silica precipitation activity.

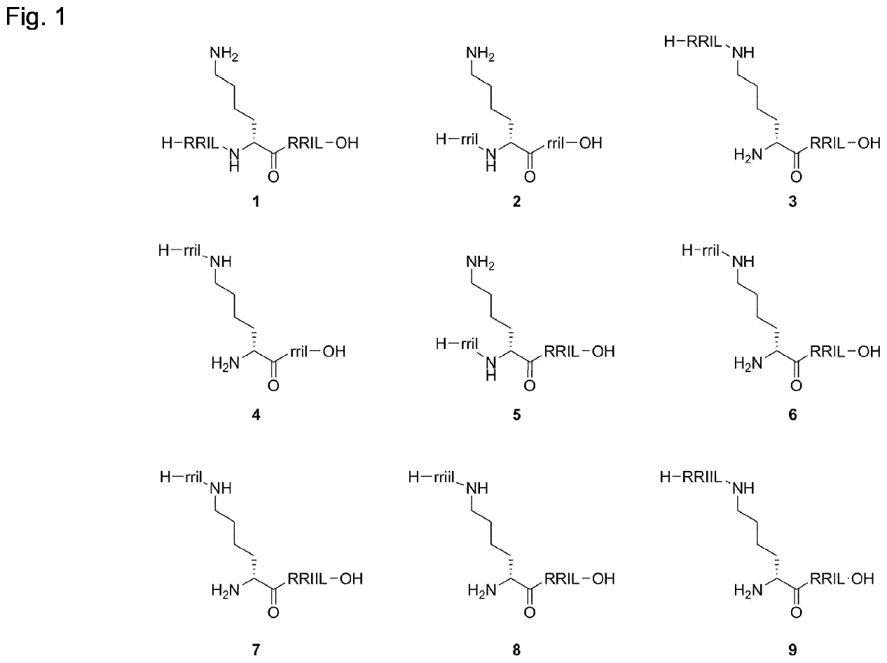

The library of peptides was derived from the RRILKRRIL sequence 1 (SEQ ID NO: 6; cf., FIG. 1). The connection points of individual monomer RRIL units (SEQ ID NO: 4) were varied between the α- and ε-amines of the central lysine residue. Inverting the stereochemistry of individual RRIL units (SEQ ID NO: 4) further diversified the peptide library. The secondary structures and potential self-assembly effects of the peptides were probed by circular dichroism (CD) whereas the morphologies of the resulting silica materials were analysed by scanning and transmission electron microscopies (SEM and TEM, respectively). We discovered that when treated with silicic acid (freshly generated from TMOS), all of the peptides showed the capacity to precipitate silica at pH 7 and in a short period of time. Electron microscopy of the silica precipitates revealed formation of ordered silica particles, with one peptide forming rod-like structures while the others precipitating spherical particles as seen for many other R5 derived peptides (C. C. Lechner and C. F. Becker, Mar. Drugs, 2015, 13, 5297-5333).

### Peptide Synthesis

The peptide library was prepared by using Fmoc-based solid phase peptide synthesis (SPPS) with standard, differentially protected building blocks of D- and L-amino acid moieties. Following synthesis, peptides were cleaved under standard conditions and purified with RP-HPLC. Peptides 3 (according to SEQ ID NO: 7), 4, 6, 7 and 8 have the RRIL (SEQ ID NO: 4) monomers connected via the α- and ε-amine of the central lysine and either D- or L-enantiomers of Boc-Lys(Fmoc)-OH were used during synthesis of these peptides with subsequent elongation with RRIL units (SEQ ID NO: 4) with respective stereochemistry. Peptides 2 and 4, comprising exclusively D-amino acid moieties, necessitated the use of the Fmoc-D-Leu-WANG resin.

All peptides were synthesized manually on solid support using fluorenylmethoxycarbonyl (Fmoc) chemistry (Atherton E, Sheppard RC. Solid Phase Synthesis: A Practical Approach. IRL Press at Oxford Univ. Press: Oxford, 1989). Syntheses were performed on 0.05 mmol scale using either Fmoc-Leu-Wang-PS resin or Fmoc-D-Leu-Wang-PS resin (Novabiochem). Deprotection of the N-terminal Fmoc-group was achieved by treating the resin twice with 20% (v/v) piperidine in DMF for 3 and 10 min, respectively.

For each amino acid coupling 5 eq. of the corresponding Fmoc-amino acid were activated with 4.5 eq. 1-[Bis(dimethylamino)methylene]-1H-1,2,3-triazolo[4,5-b]pyridinium 3-oxid hexafluorophosphate (HATU) and 10 eq. DIEA for 30 seconds and added to the resin for 20 min. At times coupling and deprotection reactions were checked by ninhydrin tests (V. K. Sarin, S. B. Kent, J. P. Tam and R. B. Merrifield, Anal. Biochem., 1981, 117, 147-157). Between coupling and deprotection steps the peptidyl-resin was washed with DMF. After completion of peptide elongation the N-terminal Fmoc group was removed, the peptidyl resin was washed with DCM and MeOH and dried under vacuum.

Overall deprotection and cleavage of peptides from resin were achieved with 2.5% TIS, 2.5% H₂O and 95% TFA for 2 hours at room temperature. Crude peptides were precipitated by addition of cold diethylether and subsequent centrifugation. Precipitated peptides were washed twice with ether and after removal of the supernatant dissolved in 20% acetonitrile in water and finally lyophilized.

Calculation of yields for purified peptides are based on the syntheses scales.

Crude peptides were purified by RP-HPLC using a preparative Kromasil C18 column and a 3%/min gradient of ACN 5-65% at 10 mL/min and the fractions were analysed by electrospray ionization mass spectrometry (ESI-MS). Analyses of the purified peptides were carried out using an analytical Kromasil C18 column on samples dissolved in 6M Guanidine-HCl, with 3%/min gradient of ACN 5-65% at 1 mL/min and UV measurement at 214 nm, both of which were commenced 5 minutes after the injections.

### Synthesis of Peptide 1 (According to SEQ ID NO: 6)

Peptide 1 was synthesised using standard SPPS procedures as described above. 23 mg of pure peptide were obtained from 19 μmol of peptidyl resin (38% yield). Analytical HPLC showed a high purity of the peptide 1. ESI-MS of purified peptide 1 showed calculated [M+2H]²⁺: 612.5, [M+3H]³⁺: 408.7.

### Synthesis of Peptide 2

Peptide 2 was synthesised using standard SPPS procedures as described above with use of D-amino acid building blocks. 18 mg of pure peptide were obtained from 15 μmol of peptidyl resin (29% yield). Analytical HPLC showed a high purity of the peptide 2. ESI-MS of purified peptide 2 showed calculated [M+2H]²⁺: 612.5, [M+3H]³⁺: 408.7.

### Synthesis of Peptide 3 (According to SEQ ID NO: 7)

Peptide 3 was synthesised using standard SPPS procedures as described above, with use of Boc-Lys(Fmoc)-OH for the coupling of the central lysine residue. 14 mg of pure peptide were obtained from 11 μmol of peptidyl resin (23% yield). Analytical HPLC showed a high purity of the peptide 3. ESI-MS of purified peptide 3 showed calculated [M+2H]²⁺: 612.5, [M+3H]³⁺: 408.7.

### Synthesis of Peptide 4

Peptide 4 was synthesised using standard SPPS procedures as described above, with use of D-amino acid building blocks and Boc-D-Lys(Fmoc)-OH for the coupling of the central lysine residue. 21 mg of pure peptide were obtained from 17 μmol of peptidyl resin (34% yield). Analytical HPLC showed a high purity of the peptide 4. ESI-MS of purified peptide 4 showed calculated [M+2H]²⁺: 612.5, [M+3H]³⁺: 408.7.

### Synthesis of Peptide 5

Peptide 5 was synthesised using standard SPPS procedures as described above with use of standard and D-amino acid building blocks. 11 mg of pure peptide were obtained from 9 μmol of peptidyl resin (18% yield). Analytical HPLC showed a high purity of the peptide 5. ESI-MS of purified peptide 5 showed calculated [M+2H]²⁺: 612.5, [M+3H]³⁺: 408.7.

### Synthesis of Peptide 6

Peptide 6 was synthesised using standard SPPS procedures as described above with use of standard and D-amino acid building blocks and Boc-Lys(Fmoc)-OH for the coupling of the central lysine residue. 17 mg of pure peptide were obtained from 14 μmol of peptidyl resin (28% yield). Analytical HPLC showed a high purity of the peptide 6. ESI-MS of purified peptide 6 showed calculated [M+2H]²⁺: 612.5, [M+3H]³⁺: 408.7.

Synthesis of peptide 7

Peptide 7 was synthesized using standard SPPS procedures as described above with use of standard and D-amino acid building blocks and Boc-Lys(Fmoc)-OH for the coupling of the central lysine residue. 9 mg of pure peptide were obtained from 7 μmol of peptidyl resin (13% yield). Analytical HPLC showed a high purity of the peptide 7. ESI-MS of purified peptide 7 showed calculated [M+2H]²⁺: 668.8 [M+3H]³⁺: 446.3.

### Synthesis of Peptide 8

Peptide 8 was synthesized using standard SPPS procedures as described above with use of standard and D-amino acid building blocks and Boc-Lys(Fmoc)-OH for the coupling of the central lysine residue. 12 mg of pure peptide were obtained from 9 μmol of peptidyl resin (18% yield). Analytical HPLC showed a high purity of the peptide 8. ESI-MS of purified peptide 8 showed calculated [M+2H]²⁺: 668.8 [M+3H]³⁺: 446.3.

### Synthesis of Peptide 9 (According to SEQ ID NO: 8)

Peptide 9 was synthesized using standard SPPS procedures as described above with use of standard amino acid building blocks and Boc-Lys(Fmoc)-OH for the coupling of the central lysine residue. 17 mg of pure peptide were obtained from 13 μmol of peptidyl resin (25% yield). Analytical HPLC showed a high purity of the peptide 9. ESI-MS of purified peptide 9 showed calculated [M+2H]²⁺: 668.8 [M+3H]³⁺: 446.3.

### Silica Precipitation

The individual members of the peptide library were subjected to silica mineralisation conditions analogous to those used for the R5 peptide (C. C. Lechner and C. F. W. Becker, Chem. Sci., 2012, 3, 3500). Phosphate buffered solution at pH 7 was used as a background for the reaction, and peptides were individually incubated in this buffer for 24 h prior to silica precipitation. White precipitate was observable in all samples within the first five minutes following addition of the silicic acid.

Peptides were dissolved to a final concentration of 0.5 mg/mL in 50 mM potassium phosphate buffer at pH 7.0 and left standing at room temperature overnight. Silicic acid was generated by hydrolysis of 250 mM tetramethoxysilane in 1 mM aqueous HCl for 4 min. Silica precipitation reactions were initiated by addition of silicic acid to peptide solutions to a final concentration of 25 mM. Reactions were incubated at RT for 30 min. Silica precipitates were collected by centrifugation (5 min, 16.873×g) and washed twice with water. Silicic acid solutions without silaffin peptides did not lead to the formation of any precipitate. All precipitations were carried out in triplicates.

### SEM Imaging

Silica precipitate collected by centrifugation was suspended in water, applied to a Thermanox™ coverslip (Thermo scientific) and air dried. Peptide samples without silica were applied onto the coverslips from phosphate buffer solutions and whisk-washed on the slides. The coverslips were placed onto sample holders and sputter coated with gold in high vacuum (Bal-Tec SCD 005). Electron micrographs were recorded with a scanning electron microscope (JEOL JSM 5900 LV) operating at 20 kV.

### TEM Imaging

Peptide and peptide-silica mixtures suspended in 50 mM phosphate buffer were applied onto carbon coated copper grid and subsequently viewed with Philips CM200 electron microscope operated at 200 kV.

### Circular Dichroism

CD spectra were recorded on peptide solutions at 0.8 mM (by weight) in 50 mM phosphate buffer at pH 7. Solutions had been incubated at room temperature for a minimum of 24 h. Scans were performed in 1 nm increments with 3 s scans at 20° C. and averaged over 5 scans.

The CD spectrum of peptide 1 in the A range of 200-250 nm was fitted by use of the novel Beta Structure Selection algorithm with normalized root mean square deviation of 0.018 (A. Micsonai, F. Wien, L. Kernya, Y.-H. Lee, Y. Goto, M. Réfrégiers and J. Kardos, Proc. Natl. Acad. Sci., 2015, 112, E3095-E3103). The algorithm predicts that the secondary or tertiary structure of the peptide consists of approximately 56% random coils, 27% beta-sheets and 17% beta-turns.

### RESULTS AND DISCUSSION

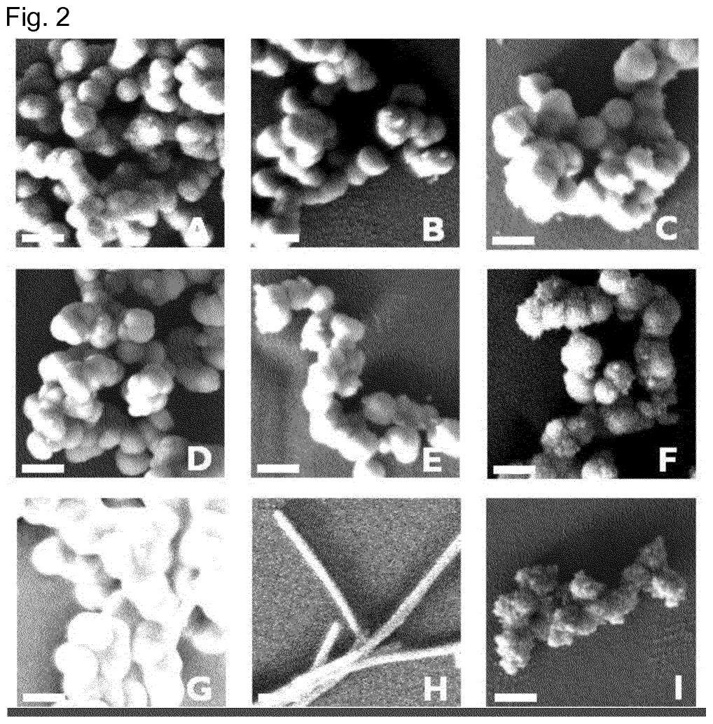

FIG. 2 shows scanning electron micrographs of the silica particles resulting from the procedure described above. The morphologies of the precipitates varied between spheres and rods. Peptides 1 (according to SEQ ID NO: 6) and 2, which are mirror images of one another, led to formation of aggregated spherical particles with diameters of 300-600 nm (FIGS. 2A and 2B, respectively), similar to previously observed particles generated with an R5 peptide (C. C. Lechner and C. F. W. Becker, Chem. Sci., 2012, 3, 3500; R. Wieneke, A. Bernecker, R. Riedel, M. Sumper, C. Steinem and A. Geyer, Org. Biomol. Chem., 2011, 9, 5482). Raspberry-like budding is noticeable for most of these samples and can be attributed to Stober-type growth of small particle aggregates, which is frequently observed for silica mineralisation that are directed by organic substances (W. Stöber, A. Fink and E. Bohn, J. Colloid Interface Sci., 1968, 26, 62-69). The N-terminal RRIL unit (SEQ ID NO: 4) is attached to the ε-amine of the central lysine of the peptide 3 (according to SEQ ID NO: 7), which allowed us to probe the impact of the distance difference between the two RRIL units (SEQ ID NO: 4) on the peptide's mineralising properties. The resulting silica particles displayed morphological similarity to particles from peptides 1 (according to SEQ ID NO: 6) and 2 (FIG. 2C). Peptide 4, which is a D-enantiomer of 3 (according to SEQ ID NO: 7), resulted in precipitates as bigger aggregates of more diverse particle sizes (FIG. 2D). In peptide 5 the N-terminal RRIL unit (SEQ ID NO: 4) consists of D-amino acid moieties while the C-terminal unit is all L. This peptide resulted in silica particles with morphologies similar to those of silica precipitates with peptides 1 (according to SEQ ID NO: 6), 2 and 3 (according to SEQ ID NO: 7) (FIG. 2E). Peptide 6 is an analogue of peptide 5 with the N-terminal RRIL (SEQ ID NO: 4) also consisting of D-amino acid moieties but here the unit is attached to ε-amine of the central lysine. This peptide led to formation of spherical particle aggregates with more uneven and irregular surfaces (FIG. 2F).

The peptide library was further diversified by adding an isoleucine moiety residue in the C-terminal and N-terminal RRIL units (SEQ ID NO: 4; peptides 7 and 8, respectively). Such an additional hydrophobic residues influences the assembly properties and it has been shown for sequences such as IIIK that specific structures such as rods can be formed (S. Wang, J. Xue, X. Ge, H. Fan, H. Xu and J. R. Lu, Chem. Commun., 2012, 48, 9415). Here, silica particles resulting from peptide 7 were similar in morphology to particles formed by other peptides in the library (spherical particles, FIG. 2G). However, spheres formed in the presence of 7 have an amorphous character and contain larger particle clusters. Peptide 8 formed silica precipitates with a very different rod-shaped morphology, suggesting a different pre-assembly of this branched construct, which contains an additional D-isoleucine moiety on the N-terminal arm of the construct. The importance of the stereochemistry is further demonstrated by peptide 9 (according to SEQ ID NO: 8), which does not lead to formation of rods even though it is an all L-analogue of 8. As seen in FIG. 2H, 8 induced silica precipitation gives rise to rods of several micrometres in length and diameters of 90-220 nm. Silica particles with peptide 9 9 (according to SEQ ID NO: 8) displayed a particularly disorganised surface morphology, with significantly less defined spherical particles and no rod structures.

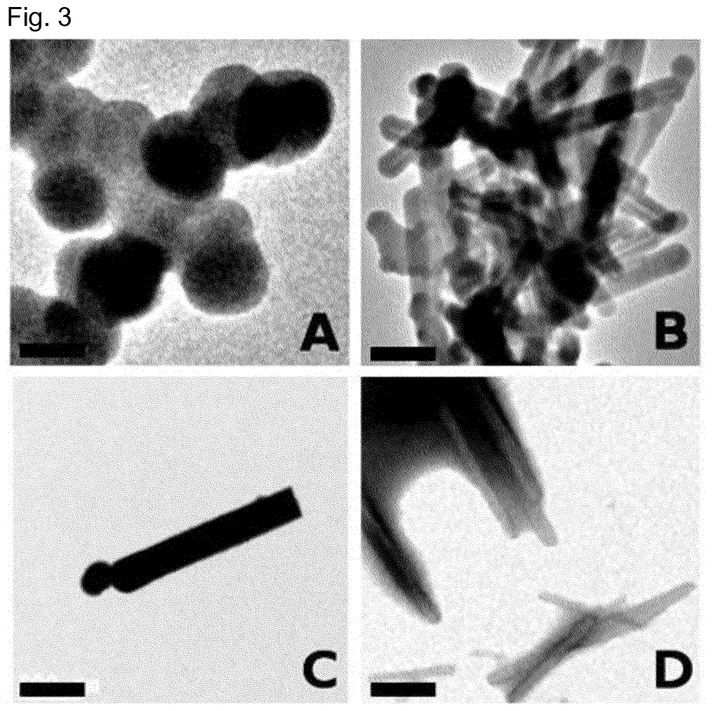

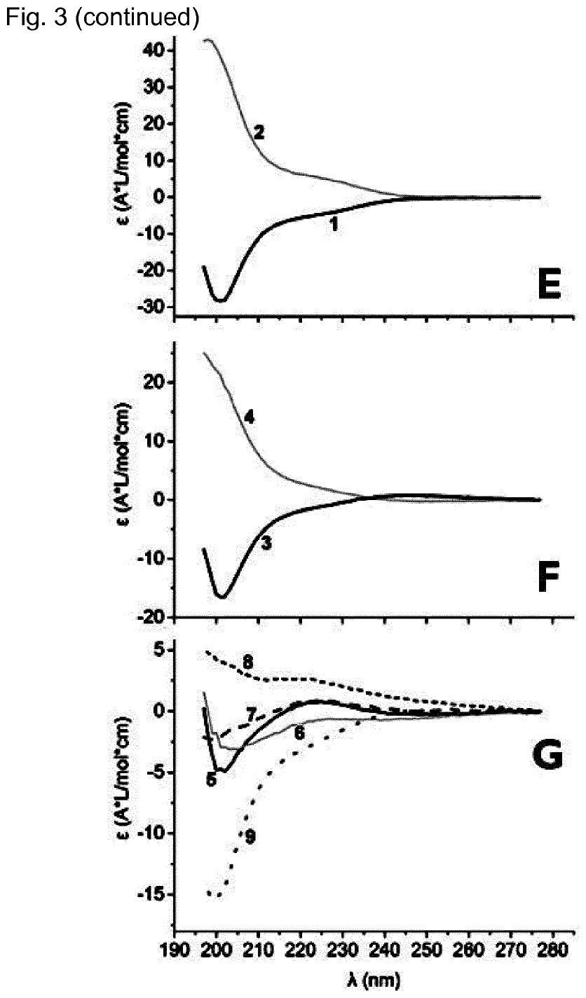

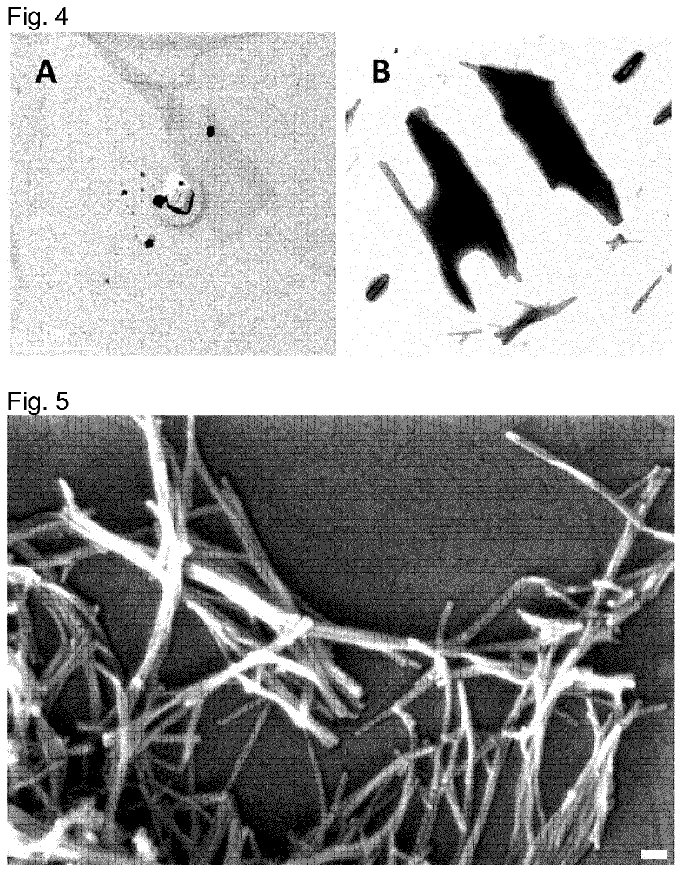

Samples from buffered peptide solutions and the silica precipitates were also investigated with TEM (FIG. 3A-D). Particles from linear peptide 1 (according to SEQ ID NO: 6) were used as representatives of all spherical particles and their morphologies were compared to structures formed in the presence of the branched, mixed D- and L-peptide 8. TEM of the buffered solution of 1 (according to SEQ ID NO: 6) without the addition of silicic acid did not show any noticeable structures apart from minor quantities of phosphate crystals (FIG. 4). Based on this finding, 1 (according to SEQ ID NO: 6) either does not form assemblies in the absence of silicic acid or possibly these assemblies are too small to be visualized by this measurement. Silica particles of 1 showed morphology similar to that observed via SEM, i.e. clusters of spherical particles (FIG. 3A). The internal density of these particles is homogeneous with higher contrast in the regions with stacked particles. Rods can be seen in the micrographs of the silica precipitation product of 8 (according to SEQ ID NO: 8; FIG. 3B, C). These particles contain an inner core of lower density, which could be explained if silicification was templated by already assembled peptide rods. TEM images of 8 in phosphate buffer in the absence of silicic acid show micrometre long rods or narrow sheets with different diameters that could act as templates (80-200 nm, FIG. 3D). Rods with wider diameters appear to be composed of narrower rods, which can indicate a continued super-assembly of the peptide (FIG. 4). Short peptide amphiphiles, such as IIIK, are known to form stable nanostructures in aqueous solutions with morphologies ranging from spherical to rod-like (S. Wang, J. Xue, X. Ge, H. Fan, H. Xu and J. R. Lu, Chem. Commun., 2012, 48, 9415).

These peptides can also be employed as templates for silica deposition under slow and low-yielding aqueous conditions (S. Wang, J. Xue, Y. Zhao, M. Du, L. Deng, H. Xu and J. R. Lu, Soft Matter, 2014, 10, 7623-7629). In our case the extra isoleucine moiety residue on the N-terminal RRIL unit of 8 increases its amphiphilic nature and thereby facilitated formation of rod structures as observed for IIIK (according to SEQ ID NO: 14). Interestingly, the addition of L-isoleucine moiety to the C-terminal RRIL unit (SEQ ID NO: 4) did not result in similar self-assembly (peptide 7), which points to a more complex assembly mechanism.

To investigate such assembly routes, secondary structure of 1-9 was assessed in phosphate buffer by circular dichroism following 24 h incubation (FIG. 3E-F). The CD spectrum of 1 (according to SEQ ID NO: 6) points towards a random coil conformation and a fitting algorithm indicated a mixture of random coil and beta turn (see Supporting Information) (A. Micsonai, F. Wien, L. Kernya, Y.-H. Lee, Y. Goto, M. Réfrégiers and J. Kardos, Proc. Natl. Acad. Sci., 2015, 112, E3095-E3103). As expected, a CD spectrum of 2 showed a mirror image of the spectrum of peptide 1 (according to SEQ ID NO: 6). Connection of the RRIL motif (SEQ ID NO: 4, in L or D) via the lysine side-chain did not impact the CD spectra of peptides 3 and 4, which showed similar profiles to the spectra of 1 (according to SEQ ID NO: 6) and 2, respectively. Since peptides 5, 6, 7 and 8 are composed of D- and L-RRIL units (SEQ ID NO: 4), the respective CD spectra have significantly lower magnitudes than those for peptides 1-4. Peptides 5 and 6 showed analogous profiles, with lower magnitudes for the latter. Inclusion of an additional isoleucine moiety in the C-terminal arm of peptide 7 did not significantly impact the CD profile as compared to peptides 5 and 6. This result was also correlated by spherical nature of the particles resulting from peptide 7. Changes were more apparent with 8, where the additional D-isoleucine moiety was installed on the N-terminal arm of the peptide. This addition influenced the peptide CD spectrum so that the minimum at 200 nm disappeared and the curve gained a positive character. This delicate change may indicate that 8 indeed folded in a manner different from other constructs and especially from peptides 6 and 7. The difference between peptides 6 and 7 is similar to that of peptides 6 and 8. Yet, only the latter case, with an addition of D-Ile particularly into the N-terminal rril arm of the construct, results in a significant change in the CD spectrum. This addition is subsequently responsible for the changes in particle morphologies from spherical to rod-like as seen in the micrographs of both free peptides and the silica precipitate. In addition, the CD profile of an all-L peptide analogue of 8, namely the construct 9 9 (according to SEQ ID NO: 8), showed that this peptide assembles in a manner largely similar to the previous peptides as the profile was similar to that of peptides 1 (according to SEQ ID NO: 6) and 3 (according to SEQ ID NO: 7).

The linear and branched constructs based on the RRIL motif (SEQ ID NO: 4) and the resulting silica particles described here have promising perspectives as novel biomaterials. The morphologies of the resulting silica particles can be slightly altered by changes in the RRIL chain (SEQ ID NO: 4) stereochemistry and orientation towards each other. However, dramatic changes have been achieved by introducing one single isoleucine moiety residues in specific D-motif, which resulted in the assembly of peptide rods that can template silica rod formation. Currently underway are further studies of the biophysical processes underlying peptide assembly and particle formation, as well as cellular uptake of particles and strategies for cargo encapsulation and delivery. The latter points could be the basis for broad biotechnological applications as it was demonstrated that silaffin peptides can be efficiently released from silica particles using pH or redox triggers (C. C. Lechner and C. F. W. Becker, Bioorg. Med. Chem., 2013, 21, 3533-3541). Furthermore, such silaffin motifs can control silica formation even in the context of much larger biomolecules such as functional proteins, leading to their encapsulation into silica under physiological conditions (C. C. Lechner and C. F. Becker, Biomater. Sci., 2015, 3, 288-297).

Summarized, most branched and linear combinations of RRIL (SEQ ID NO: 4) resulted in spherical particles, while one branched combination of a D- and L-RRIL motif (SEQ ID NO: 4), formed silica rods. The biomimetic peptide-silica particles and rods described here have promising perspectives as novel composite materials for use in biological systems.

### Example 2—Testing Further Peptides Comprising Alternative Motifs According of the Present Invention for Preparing Silica Particles

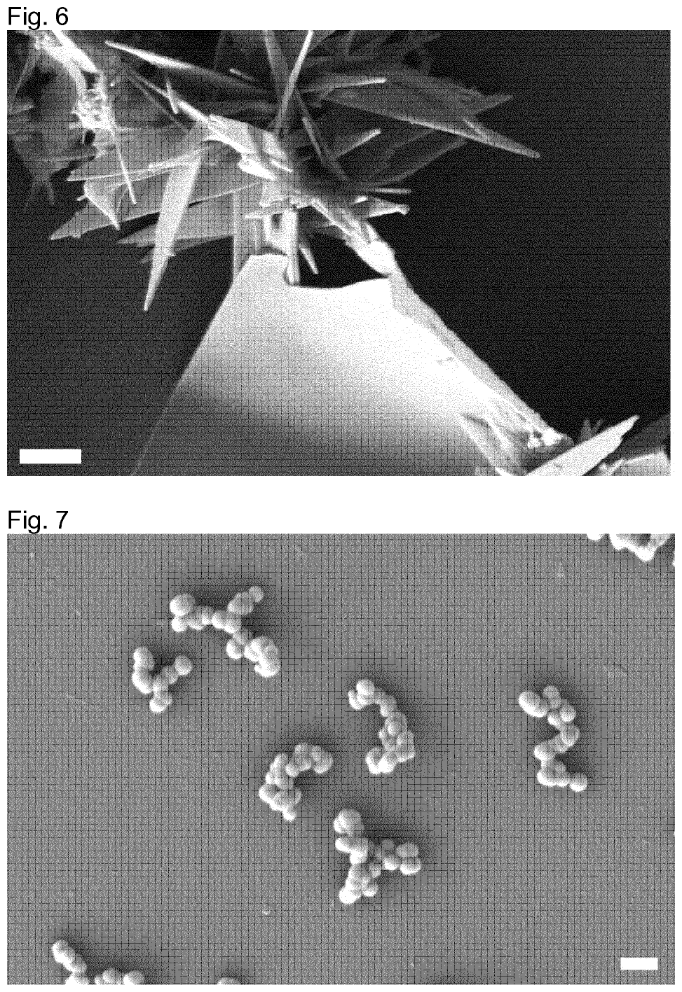

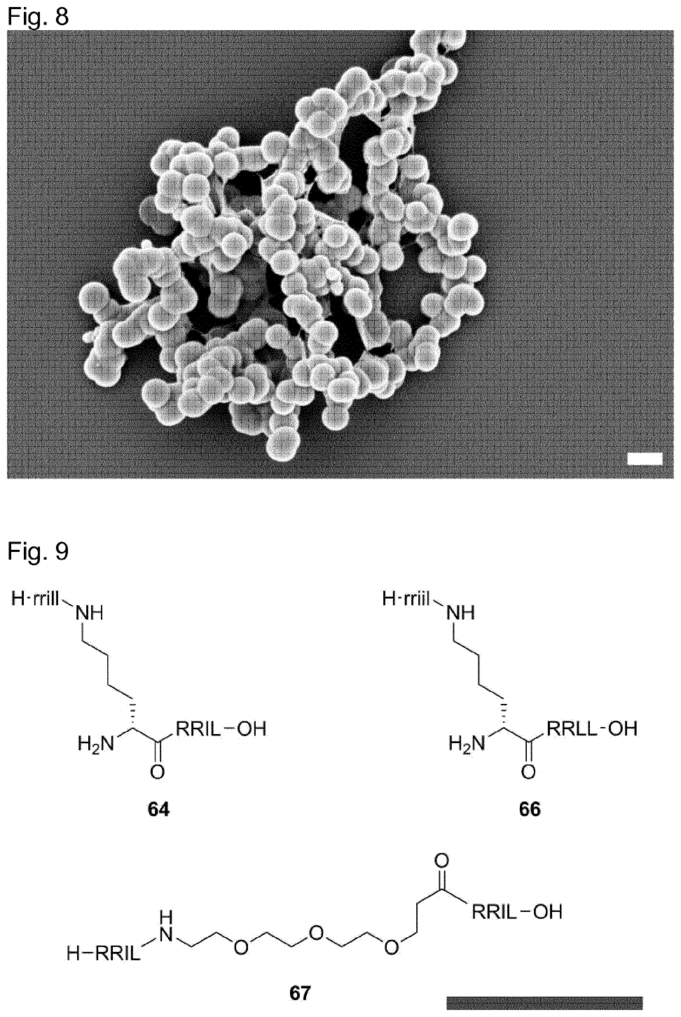

Peptides 64 and 66 (FIG. 9) extend the scope of the morphologies of the silica material precipitated by the silaffin peptides. In the case of peptide 64, presence of an extra leucine residue in position 4 leads to the formation of silica with fibrillar morphology (FIG. 5). In peptide 66, the N-terminal peptide lacks isoleucine residues altogether and it is still able to precipitate silica, in this case with sheet-like morphology (FIG. 6).

### Example 3—Testing a Further Peptide Comprising an Alternative Linker Moiety for Preparing Silica Particles

Peptide 67 consists of two RRIL motifs connected via a triethylene glycol chain. This assembly demonstrates that, instead of a lysine residue in position 5, also an alternative linker such as a polyethylene glycol linker moiety may be used. This peptide also precipitates ordered silica particles (FIG. 7).

### Example 4—Vaccine Conjugate of the R5 Peptide

Peptide 68 (SIINFEKLCSSKKSGSYSGSKGSKRRIL (SEQ ID NO: 32) represents a conjugate of the R5 peptide with an ovalbumin epitope, commonly used for vaccine evaluation. This construct proves that a vaccine antigen can be covalently attached to the R5 peptide and that such an assembly leads to similar silica morphologies as the R5 alone. The resulting ordered spherical silica particles are depicted in FIG. 8.

1. A vaccine composition V comprising
(A) a peptidic entity A comprising at least one sequence motif A1 of a sequence
(SEQ ID NO: 23)
RR-X^{1a}-X^{1b}-L,
wherein
X^{1a} is a hydrophobic amino acid moiety,
X^{1b} is a peptide bond or a hydrophobic amino acid moiety,
or a D-peptide analogue of the sequence SEQ ID NO: 23, wherein
one or more of the L-amino acid moieties is/are replaced by the respective D-amino acid(s),
or a retro-inverso analogue of the sequence of SEQ ID NO: 23,
or a peptidomimetic analogue of the sequence of SEQ ID NO: 23;
(B) an antigen of interest B; and
(C) silica particles C embracing the peptidic entity A and the antigen of interest B; and
(D) optionally a pharmaceutically acceptable carrier.

2. The vaccine composition V of claim 1, wherein the peptidic entity A and the antigen of interest B together do not comprise more than 50 amino acid moieties, preferably not more than 40 amino acid moieties, in particular not more than 30 amino acid moieties.

3. The vaccine composition V of any of claim 1 or 2 comprising
(A) a peptidic entity A comprising:
(A1) at least one first sequence motif A1 of a sequence
(SEQ ID NO: 23)
RR-X^{1a}-X^{1b}-L,
wherein
X^{1a} is a hydrophobic amino acid moiety,
X^{1b} is a peptide bond or a hydrophobic amino acid moiety,
or a D-peptide analogue of the sequence SEQ ID NO: 23,
wherein one or more of the L-amino acid moieties is/are replaced by the respective D-amino acid(s),
or a retro-inverso analogue of the sequence of SEQ ID NO: 23,
or a peptidomimetic analogue of the sequence of SEQ ID NO: 23; and
(A2) at least one second sequence motif A2 of a sequence selected from the group consisting of
(SEQ ID NO: 23)
RR-X^{1a}-X^{1b}-L,
and
(SEQ ID NO: 24)
L-X^{1c}-X^{1d}-RR,
wherein
X^{1a} is a hydrophobic amino acid moiety,
X^{1b} is a peptide bond or a hydrophobic amino acid moiety,
X^{1c} is a hydrophobic amino acid moiety,
X^{1d} is a bond or a hydrophobic amino acid moiety,
or a D-peptide analogue of the sequence SEQ ID NO: 23 or 24, wherein one or more of the L-amino acid moieties is/are replaced by the respective D-amino acid(s),
or a retro-inverso analogue of the sequence of SEQ ID NO: 23 or 24,
or a peptidomimetic analogue of the sequence of SEQ ID NO: 23 or 24;
(B) an antigen of interest B; and
(C) silica particles C embracing the peptidic entity A and the antigen of interest B; and
(D) optionally a pharmaceutically acceptable carrier.

4. The vaccine composition V of any of claims 1 to 3, wherein:
at least one X^{1a} is an isoleucine moiety;
X^{1c} is an isoleucine moiety;
at least one X^{1b} is a peptide bond or isoleucine moiety; and/or
X^{1c} is a peptide bond or isoleucine moiety.

5. The vaccine composition V of any of claims 1 to 4, wherein the peptidic entity A comprises at least one sequence motif A12 selected from the group consisting of:
(SEQ ID NO: 25)
RR-X^{1a}-X^{1b}-L-X²-RR-X^{3a}-X^{3b}-L
(SEQ ID NO: 26)
L-X^{1a}-X^{1b}-RR-X²-RR-X^{3a}-X^{3b}-L,
and
(SEQ ID NO: 27)
RR-X^{3a}-X^{3b}-L-X²-L-X^{1a}-X^{1b}-RR,
wherein
X^{1a} and X^{1b} each independently from another have the same meaning as defined in any of claims 1 to 4,
X³² and X^{3b} each independently from another have the same meaning as X^{1a} and X^{1b} as defined in any of claims 1 to 4, and
X² is a linker moiety of up to 100 carbon atoms,
or a D-peptide analogue of the sequence SEQ ID NO: 25, 26 or 27, wherein one or more of the L-amino acid moieties is/are replaced by the respective D-amino acid(s),
or a retro-inverso analogue of the sequence of SEQ ID NO: 25, 26 or 27,
or a peptidomimetic analogue of the sequence of SEQ ID NO: 25, 26 or 27, preferably wherein X² is a peptide moiety of not more than five consecutive amino acid moieties, more preferably wherein X² comprises or consists of K or εK optionally substituted by a sequence of four or five consecutive amino acid moieties conjugated to the second amino group of the lysine moiety.

6. The vaccine composition V of any of claims 1 to 5, wherein the sequence motif A12 is selected from the group consisting of:
(SEQ ID NO: 6)
RRILKRRIL;
(SEQ ID NO: 7)
RRIL(εEK)RRIL;
(SEQ ID NO: 8)
RRIIL(εK)RRIL;
(SEQ ID NO: 9)
RRILKRRIIL;
(SEQ ID NO: 10)
RRIILKRRIL;
(SEQ ID NO: 11)
RRIILKRRIIL;
(SEQ ID NO: 12)
RRIL(εK)RRIIL;
(SEQ ID NO: 13)
RRIIL(εK)RRIIL;
(SEQ ID NO: 16)
LIRRKRRIL;
(SEQ ID NO: 17)
LIRR(εK)RRIL;
(SEQ ID NO: 18)
RRILKLIIRR;
(SEQ ID NO: 29)
RRILL(εK)RRIL;
(SEQ ID NO: 30)
RRIIL(εK)RRLL;
(SEQ ID NO: 31)
RRIL(PEG)₃RRIL;
(SEQ ID NO: 28)
LIRRK(X⁴)RRIL,
wherein X⁴ is a sequence of four or five consecutive amino acid moieties conjugated to the epsilon amino group of the lysine moiety, preferably is a sequence of SEQ ID NO: 23 or SEQ ID NO: 24;
and a D-peptide analogue of any of these sequences of SEQ ID NO: 6-13, 16-18 or 28, wherein in said D-peptide analogue at least one motif RRIL (SEQ ID NO: 4) is replaced by its D-peptide analogue rril, and/or at least one motif RRIIL (SEQ ID NO: 5) is replaced by its D-peptide analogue rriil, and/or at least one motif LIRR (SEQ ID NO: 19) is replaced by its D-peptide analogue lirr, and/or at least one motif LIIRR (SEQ ID NO: 21) is replaced by its D-peptide analogue Him and/or at least one motif RRILL (SEQ ID NO: 33) is replaced by its D-peptide analogue rrill, and/or at least one motif RRLL (SEQ ID NO: 34) is replaced by its D-peptide analogue rrll,
and optionally the lysine moiety K is replaced by its D-amino acid analogue k or the lysine moiety (εK) is replaced by its D-amino acid analogue (εk),
a retro-inverso analogue thereof; and
a peptidomimetic analogue thereof.

7. The vaccine composition V of any of claims 1 to 6, wherein the antigen of interest B is selected from the group consisting of a peptidic entity, a nucleic acid entity, an oligo- or polysaccharide entity, and a combination or conjugate of two or more thereof.

8. The vaccine composition V of any of claims 1 to 7, wherein the peptidic entity A and the antigen of interest B are conjugated with another, in particular wherein the peptidic entity A and the antigen of interest B together form a peptide strand AB.

9. The vaccine composition V of any of claims 1 to 8, wherein the silica particles C have been obtained by precipitating silica from a solution comprising silicic acid in the presence of the peptidic entity A and preferably the antigen of interest B; and/or
wherein the silica particles C bear spherical or rod-like structures having mean diameters of 0.1 to 10 μm, in particular 0.5 to 2 μm.

10. A peptidic entity A comprising:
(A1) at least one first sequence motif A1 of a sequence
(SEQ ID NO: 23)
RR-X^{1a}-X^{1b}-L,
wherein
X^{1a} is a hydrophobic amino acid moiety,
X^{1b} is a peptide bond or a hydrophobic amino acid moiety,
or a D-peptide analogue of the sequence SEQ ID NO: 23, wherein one or more of the L-amino acid moieties is/are replaced by the respective D-amino acid(s),
or a retro-inverso analogue of the sequence of SEQ ID NO: 23,
or a peptidomimetic analogue of the sequence of SEQ ID NO: 23; and
(A2) at least one second sequence motif A2 of a sequence selected from the group consisting of
(SEQ ID NO: 23)
RR-X^{1a}-X^{1b}-L,
and
(SEQ ID NO: 24)
L-X^{1c}-X^{1d}-RR,
wherein
X^{1a} is a hydrophobic amino acid moiety,
X^{1b} is a peptide bond or a hydrophobic amino acid moiety,
X^{1c} is a hydrophobic amino acid moiety,
X^{1d} is a bond or a hydrophobic amino acid moiety,
or a D-peptide analogue of the sequence SEQ ID NO: 23 or 24,
wherein one or more of the L-amino acid moieties is/are replaced by the respective D-amino acid(s),
or a retro-inverso analogue of the sequence of SEQ ID NO: 23 or 24,
or a peptidomimetic analogue of the sequence of SEQ ID NO: 23 or 24;
preferably wherein the peptidic entity A is defined as in any of claims 4 to 9.

11. A silica particle C comprising silica and a peptidic entity comprising a sequence motif A12 according to claim 10, preferably wherein said silica particle C:
is obtained by precipitating silica from a solution comprising silicic acid in the presence of the peptidic entity A, and/or
bears spherical or rod-like structures having mean diameters of 0.1 to 10 μm, in particular 0.5 to 2 μm,
in particular wherein said silica particle C is a silica particle C according to any of claims 1 to 9.

12. Use of a vaccine composition V according to any of claims 1 to 9 for vaccination.

13. A method for preparing a vaccine composition V according to any of claims 1 to 9, comprising the following steps:
(i) providing the following:
(a) a peptidic entity A according to any of claims 1 to 6,
(b) an antigen of interest B according to any of claim 1 or 7, wherein said peptidic entity A and said antigen of interest B may optionally be conjugated with another, and
(c) an aqueous solution SC comprising silicic acid;
(ii) dissolving the peptidic entity A and the antigen of interest B in the aqueous solution SC, thereby forming solution S;
(iii) incubating the solution S obtained from step (ii) under conditions allowing the precipitation of silica particles C embracing the peptidic entity A and the antigen of interest B;
(iv) optionally separating the silica particles C obtained from step (iii) from the solution S;
(v) optionally washing the silica particles C obtained from any of steps (iii) or (iv);
(vi) optionally drying the silica particles C obtained from any of steps (iii) to (v); and
(vii) optionally adding a pharmaceutically acceptable carrier to the silica particles C obtained from any of steps (iii) to (vi).

14. A method for preparing silica particle C according to claim 11, comprising the following steps:
(i) providing the following:
(a) peptidic entity A comprising a sequence motif A12 according to claim 10,
(b) optionally an antigen of interest B according to any of claim 1 or 7, wherein said peptidic entity A and said antigen of interest B, if present, may optionally be conjugated with another, and
(c) an aqueous solution SC comprising silicic acid;
(ii) dissolving the peptidic entity A comprising a sequence motif A12 and, if present, the antigen of interest B in the aqueous solution SC, thereby forming solution S;
(iii) incubating the solution S obtained from step (ii) under conditions allowing the precipitation of silica particles C embracing the peptidic entity A and, if present, the antigen of interest B;
(iv) optionally separating the silica particles C obtained from step (iii) from the solution S;
(v) optionally washing the silica particles C obtained from any of steps (iii) or (iv);
(vi) optionally drying the silica particles C obtained from any of steps (iii) to (v); and
(vii) optionally adding a pharmaceutically acceptable carrier to the silica particles C obtained from any of steps (iii) to (vi).

15. The vaccine composition V according to any of claims 1 to 9 or the silica particle C according to claim 11 for use as a medicament.

16. The vaccine composition V according to any of claims 1 to 9 for use in a method for preventing or treating a patient being of risk of or suffering from a pathologic condition associated with a pathogen or mutated cells bearing the antigen of interest B.

17. A method for producing an antibody of interest directed towards an antigen of interest B, said method comprising the steps:
(I) providing a non-human animal;
(II) applying a vaccine composition V comprising the antigen of interest B according to any of claims 1 to 9 to the non-human animal;
(III) keeping the non-human animal vaccinated according to step (ii) under conditions allowing a secondary immune response directed towards the antigen of interest B comprised in the vaccine composition V; and
(IV) obtaining the antibody of interest from body fluids, in particular blood, of the non-human animal of step (iii).
